(19) United States
(12) Patent Application Publication
(10) Pub. No.: US 2012/0082589 A1
(43) Pub. Date: Apr. 5, 2012

(54) Surgical Tray Sterilization Process Using A Transparent Sterilization, Storage, Display and Transportation System

(76) Inventors: Timothy J. Ladison, Williamsburg, VA (US); Christina Jo (Dindl) Snyder, Sudlersville, MD (US)

(21) Appl. No.: 13/244,649

(22) Filed: Sep. 25, 2011

Related U.S. Application Data
(63) Continuation of application No. 12/799,380, filed on Apr. 23, 2010.
(60) Provisional application No. 61/281,032, filed on Nov. 12, 2009.
(60) Provisional application No. 61/281,032, filed on Nov. 12, 2009.

(51) Int. Cl. A61L 2/20; A61L 2/18; A61L 2/00

(52) U.S. Cl. 422/26; 422/1; 422/28

(57) ABSTRACT

A method for sterilizing a set of medical instrument trays with a. set of instruments is provided. The method comprises the step of linking, selectively, transparent sterilization, storage, display and transport (SSDT) devices together and further includes the step of loading each SSDT device with a portion of the set of medical instrument trays when the lid assembly is removed and closing, sealing and latching each loaded SSDT device individually with the transparent lid assembly, The method includes the step of sterilizing, simultaneously, the closed, sealed and latched SSDT devices linked together so that the portion of the set of medical instrument trays in each SSDT is visible in the interior chamber of the sterilized SSDT device through the transparent lid assembly. During sterilizing, at least two medical instrument trays in each SSDT are visible 360° in the interior chamber of the sterilized SSDT device through the transparent lid assembly.

### COPENDING APPLICATIONS

This application is a continuation and claims priority benefit of U.S. Nonprovisional patent application Ser. No. 12/799,380 entitled “A TRANSPARENT STERILIZATION, STORAGE, DISPLAY AND TRANSPORTATION SYSTEM” tiled on Apr. 23, 2010, having the same inventor of the instant patent application and which is incorporated herein by references as if set forth in full below. Furthermore, U.S. Nonprovisional patent application Ser. No. 12/799,380 claims priority benefit of U.S. Provisional Patent Application No. 61/281,032, filed Nov. 12, 2009, titled “MODULAR SURGICAL TRAY STERILIZATION AND TRANSPORT SYSTEM” having the same inventor of the instant patent application and which is incorporated herein by references as if set forth in full below

### NOTICE OF COPYRIGHT PROTECTION

A portion of the disclosure of this patent document and its figures contain material subject to copyright protection. The copyright owner has no objection to the facsimile reproduction by anyone of the patent document or the patent disclosure, but otherwise reserves all copyrights whatsoever.

### BACKGROUND

I. Field

The invention relates to a sterilization process using a transparent sterilization, storage, display and transportation (SSDT) device and system.

II. Background

Hospitals have excessive costs associated with wrapping surgical instrument trays individually both from a human resource capital and a material cost standpoint. The wrapping method has many draw backs including labor intensive, material intensive, and is subject to human error and waste. Overall, the wrapping method is generally not efficient.

For example, the wrapping method loads the medical instrument trays with the instruments and/or supplies needed for a surgical procedure, and then individually wraps each tray with a filter wrap sheet which is discarded after each use. Thus, the filter wrapping process which uses the filter wrap sheet does not promote environment conservation and provides the least secure solution for protecting sterilized surgical instruments as the filter wrap sheet has no means of protection from tears, punctures, pierces, non-sterile liquids, etc.

The filter wrap sheet is subject to damage in a variety of ways. For example, care should be taken to prevent punctures or tears from the corners or other protuberances radiating from the trays that may snag, tear or puncture the protective filter wrap sheet.

After removal of a filter wrapped tray from the autoclave, there is a potential for contamination of the sterilized trays and instruments. Currently, the sterilized instruments should be used within a set time period (hereinafter referred to as a “shelf life”) after sterilization before becoming stale, albeit the trays may have not been opened. Hospitals regularly re-sterilize many stale trays which wastes water, energy and personnel resources for re-sterilization.

In another system, surgical instrument tray containers include lids with a filter. However, these containers are not cost efficient, as each container can generally only hold a single tray. Another drawback with both the filter wrap process and the surgical instrument tray containers is that the contents of the surgical tray itself cannot be easily inspected. Some containers include two filters on the lid. The filters obscure the personnel's ability to inspect the instruments within the container.

Each surgeon requests a list of medical instruments for carrying out a particular surgical procedure. However, the personnel delivering the surgical instruments may get the trays mixed up because of the inability to visually inspect the interior of the instrument trays. When sterile instruments are erroneously opened for the wrong surgeon, the opened sterile instruments, even though not used, must be re-sterilized causing a waste in water, material and labor. Furthermore, the single tray storage containers are not readily stackable on top of one another as the steam, sterilant agent and/or heated air is not allowed adequate unobstructed ingress and egress for sterilization of the instruments within the container. Thus, the capacity of the autoclave to sterilize multiple trays is generally not efficiently utilized as stacking, in a space efficient manner, is not easily accommodated.

In another system, a wheeled cabinet configured to house therein a plurality of trays (e.g., eight or twelve trays) has been designed. However, not all surgeries require eight or twelve trays. For example, some surgeries only need four (4) trays. Typically, the wheeled cabinet has one door to access the trays and is made of stainless steel which does not allow the instruments to be viewed before opening the cabinet. Thus, the surgical team does not have full access to the trays and cannot visually inspect the instruments to minimize errors and waste before opening. Furthermore, the surgical team may waste time finding and accessing a particular tray needed of the many trays stored in the wheeled cabinet. The wheeled cabinet is not modular, customizable and re-customizable to adapt the cabinet on demand to conduct a surgery. The wheeled cabinet is bulky and takes up space in an environment where storage space is needed.

Thus, there is a need for a method of sterilization that reduces the time for sterile processing and reduces material and resource costs associated with preparing surgical instruments.

There is a need for a method that employs a system that is modular, customizable and re-customizable so that only those surgical instrument trays that are needed for surgery are sterilized, stored, displayed and transported to and from the surgery rooms in a compact and organized manner.

There is a need for a method of sterilization that allows multiple trays with instruments to be viewed 360 degrees in a single container that is sterile and secure.

### SUMMARY

The aforementioned problems, and other problems, are reduced, according to exemplary embodiments, by a sterilization process that employs a transparent sterilization, storage, display and transportation (SSDT) device and system configured to stack within each device a plurality of trays.

In an exemplary embodiment, a method for sterilizing a set of medical instrument trays with a set of instruments is provided. The method comprises the steps of: linking, selectively, transparent sterilization, storage, display and transport (SSDT) devices together wherein each SSDT device includes a transparent lid assembly having an interior chamber; loading each SSDT device with a portion of the set of medical instrument trays when the lid assembly is removed; closing, sealing and latching each loaded SSDT device individually with the transparent lid assembly; and sterilizing, simultaneously, the closed, sealed and latched SSDT devices linked together wherein the portion of the set of medical instrument trays in each SSDT is visible in the interior chamber of the sterilized SSDT device through the transparent lid assembly.

In another exemplary embodiment, a method for sterilizing a set of medical instrument trays with a set of instruments comprises the steps of: selectively linking together two or more selected transparent sterilization, storage, display and transport (SSDT) devices of a set of SSDT devices based on a number of trays in the set of medical instrument trays wherein each SSDT device includes a transparent lid assembly having an interior chamber; loading each SSDT device with two or more medical instrument trays when the lid assembly is removed; closing, sealing and latching each loaded SSDT device individually with the transparent lid assembly; and sterilizing, simultaneously, the closed, sealed and latched SSDT devices linked together wherein at least two medical instrument trays in each SSDT are visible 360° in the interior chamber of the sterilized SSDT device through the transparent lid assembly.

Other systems, methods, and/or devices according to embodiments will be or become apparent to one with skill in the art upon review of the following drawings, and further description. It is intended that all such additional systems, methods, and/or devices be included within this description, be within the scope of the present invention, and be protected by the accompanying claims.

### BRIEF DESCRIPTION OF THE DRAWINGS

The above and other exemplary embodiments, objects, uses, advantages, and novel features are more clearly understood by reference to the following description taken in connection with the accompanying figures wherein:

FIG. 1 illustrates a perspective view of a sterilization, storage, display and transportation (SSDT) device without filter covers installed in accordance with some exemplary embodiments of the present invention;

FIG. 2 illustrates an exploded view of the sterilization, storage, display and transportation (SSDT) device in accordance with some exemplary embodiments of the present invention;

FIG. 3 illustrates a partial exploded view of the configurable filtered base carriage (CFBC) assembly without wheel assemblies and from a first perspective in accordance with some exemplary embodiments of the present invention;

FIG. 4 illustrates a partial exploded view of the configurable filtered base carriage (CFBC) assembly with wheel assemblies and from a second perspective in accordance with some exemplary embodiments of the present invention;

FIG. 5A illustrates an unlocked state of a wheel assembly when attached to the base carriage platform in accordance with some exemplary embodiments of the present invention;

FIG. 5B illustrates a locked state of the wheel assembly when attached to the base carriage platform in accordance with some exemplary embodiments of the present invention;

FIG. 6 illustrates an exploded view of a wheel assembly in accordance with some exemplary embodiments of the present invention;

FIG. 7 illustrates a latch mechanism to latch the transparent filtered lid (TFL) assembly in accordance with some exemplary embodiments of the present, invention;

FIG. 8 illustrates an exploded view of a shelf assembly in accordance with some exemplary embodiments of the present invention;

FIG. 9 illustrates a partial view of a shelf wall and the base carriage platform in accordance with some exemplary embodiments of the present invention;

FIG. 10 illustrates an exploded view of the transparent filtered lid (TFL) assembly in accordance with some exemplary embodiments of the present invention;

FIG. 11A illustrates a side view of the transparent box-shaped lid structure in accordance with some exemplary embodiments of the present invention;

FIG. 11B illustrates a bottom view of the transparent box-shaped lid structure in accordance with some exemplary embodiments of the present invention;

FIG. 12A illustrates a first perspective view of the filter keeper frame in accordance with some exemplary embodiments of the present invention;

FIG. 12B illustrates a second perspective view of the filter keeper frame in accordance with some exemplary embodiments of the present invention;

FIG. 13 illustrates a transparent tray for placement and sterilization of medical instruments and other items used in surgical procedures in accordance with some exemplary embodiments of the present invention;

FIG. 14 illustrates a loaded sterilization, storage, display and transportation device for use in the transparent sterilization, storage and transportation system in accordance with some exemplary embodiments of the present invention;

FIG. 15 illustrates a flowchart of a sterilization method using a sterilization, storage, display and transportation (SSDT) system in accordance with some exemplary embodiments of the present invention;

FIG. 16 illustrates stages of a pre-sterilization phase of the method of FIG. 15;

FIGS. 17A-17B illustrates a flowchart of the sterilization phase of the method of FIG. 15;

FIGS. 18A-18C illustrate the hitching and loading of a first level SSDT devices of the sterilization, storage, display and transportation (SSDT) system in accordance with some exemplary embodiments of the present invention;

FIGS. 18D-18F illustrate the hitching and loading of a second level base carriages of the sterilization, storage, display and transportation (SSDT) system in accordance with some exemplary embodiments of the present invention;

FIG. 19 illustrates the delivery and rolling of the sterilization, storage, display and transportation (SSDT) system into the autoclave from the autoclave base carriage in accordance with some exemplary embodiments of the present invention; and

FIGS. 20A and 20B illustrate a flowchart of the sterilization process in accordance with some exemplary embodiments of the present invention.

### DESCRIPTION

The word “exemplary” is used herein to mean “serving as an example, instance, or illustration.” Any configuration or design described herein as “exemplary” is not necessarily to be construed as preferred or advantageous over other configurations or designs. Furthermore, use of the words “present invention” is used herein to convey only some of the embodiments of the invention. For example, the word “present invention” would also include alternative embodiments and equivalent systems and components that one of ordinary skill in the art understands. An example is that the materials used for the exemplary embodiments may be made out of man-made materials, natural materials, and combinations thereof. A further example is that the apparatus or components of the apparatus may be manufactured by machine(s), human(s) and combinations thereof.

Some of the embodiments of the invention now will be described more fully hereinafter with reference to the accompanying drawings, in which exemplary embodiments are shown. This invention may, however, be embodied in many different forms and should not be construed as limited to the embodiments set forth herein. These embodiments are provided so that this disclosure will be thorough and complete and will fully convey the scope of the invention to those of ordinary skill in the art. Moreover, all statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

The terms transparent and translucent may be used interchangeable. In some instances, transparent materials may be colored, tinted or clear. Translucent materials may be colored and tinted and may be less clear than a transparent material. Nonetheless, transparent and translucent provides a surface that has a see through quality or property.

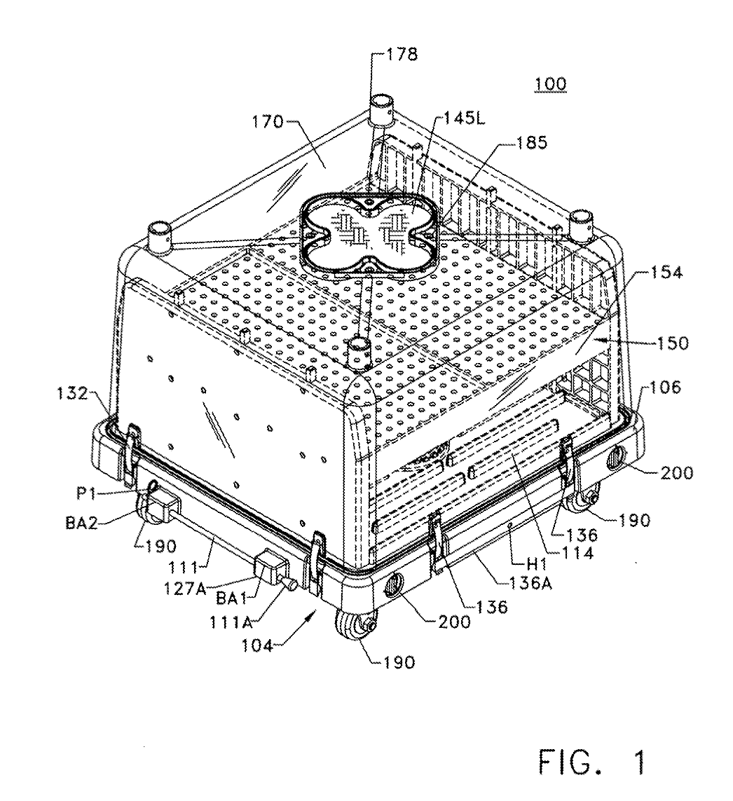

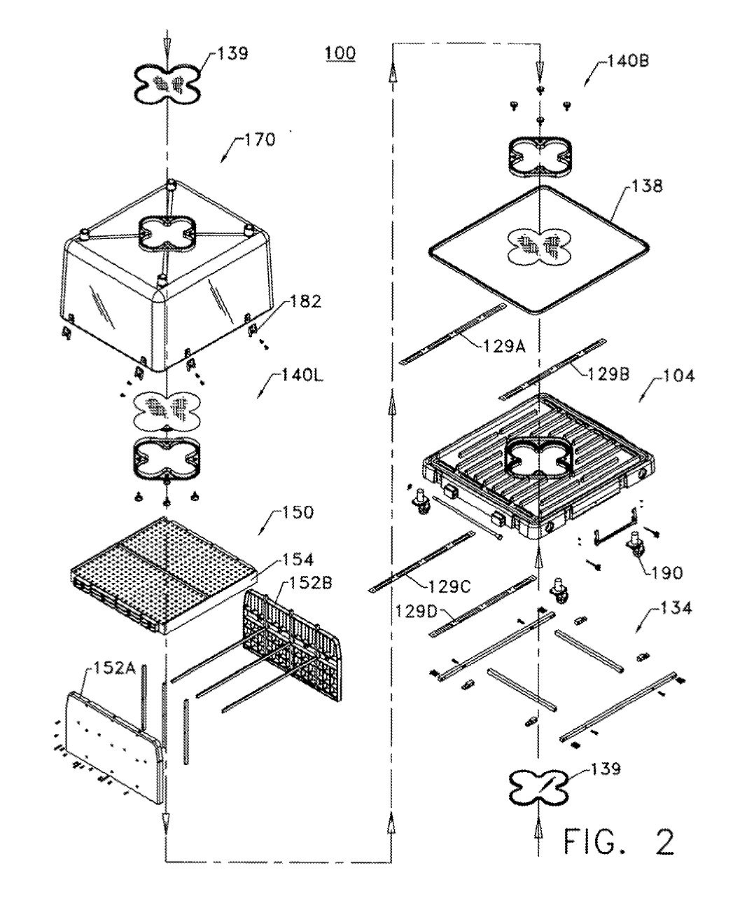

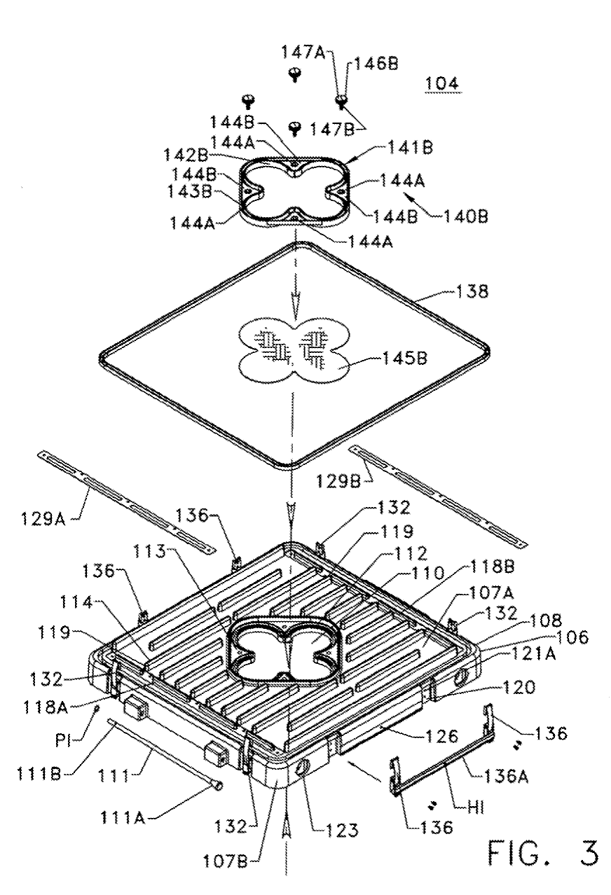

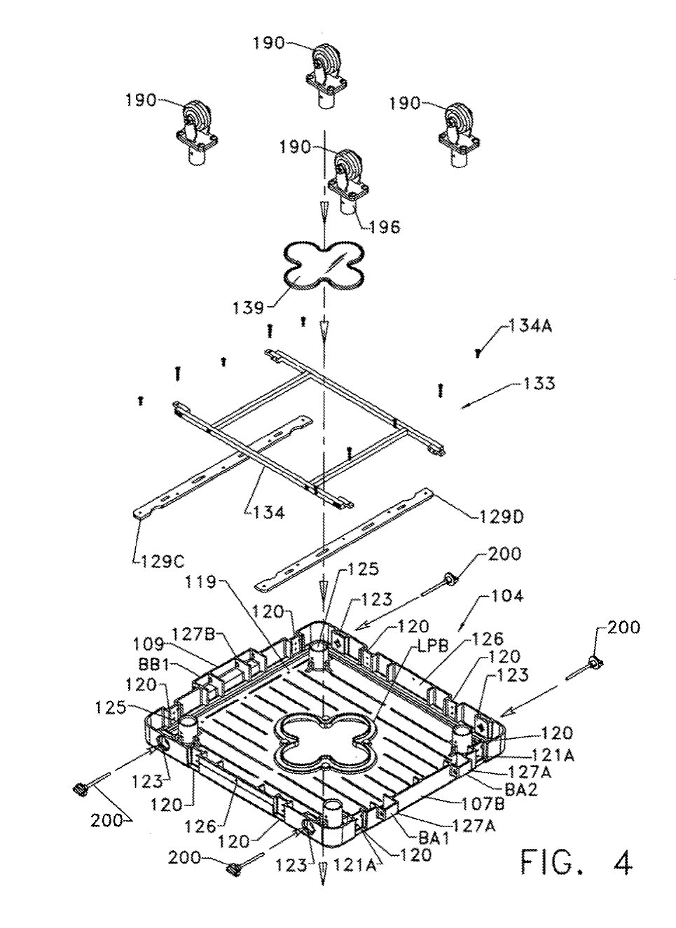

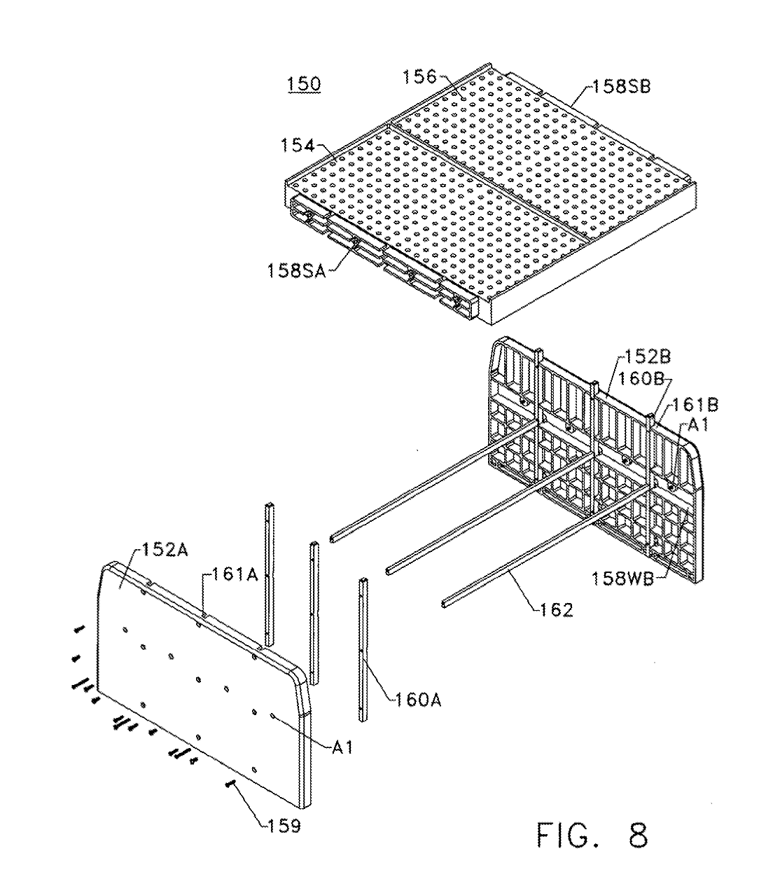

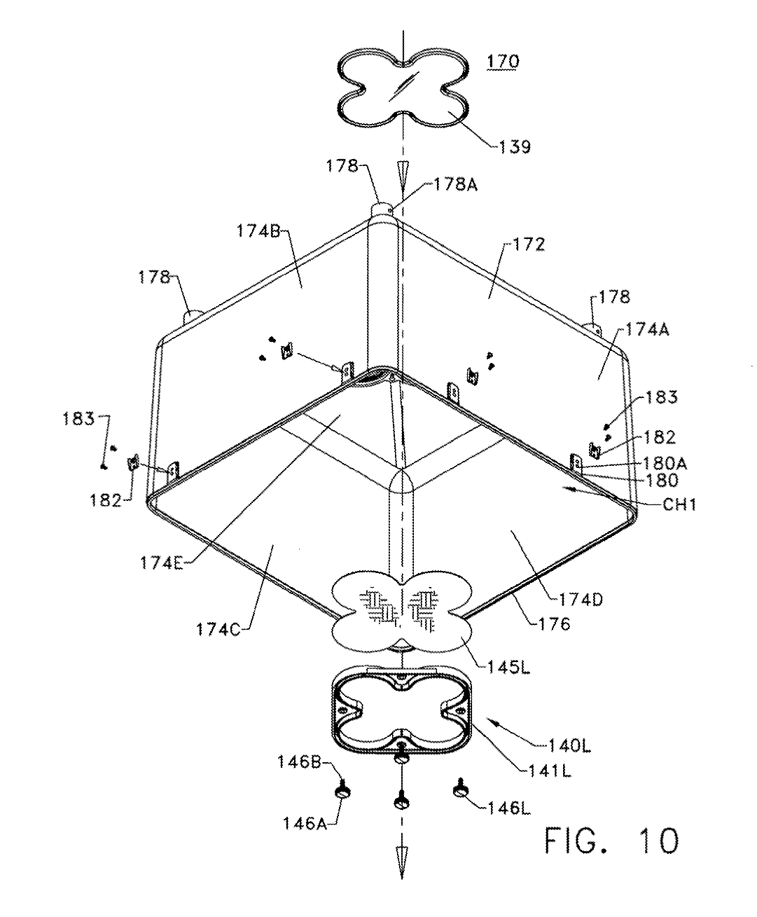

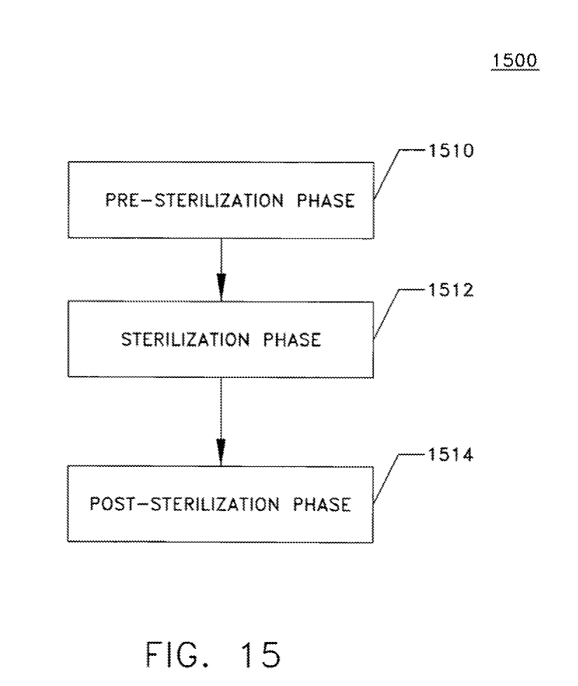

Referring now to the drawings, and in particular FIG. 1 illustrates a perspective view of a sterilization, storage, display and transportation (SSDT) device 100 without filter covers in accordance with some exemplary embodiments of the present invention. FIG. 2 illustrates an exploded view of the SSDT device 100 in accordance with some exemplary embodiments of the present invention. The SSDT device 100 includes in general a configurable filtered base carriage (CFBC) assembly 104 (FIG. 3) with a plurality of removable wheel assemblies 190 (FIGS. 4-6), a shelf assembly 150 (FIG. 8) and a transparent filtered lid (TFL) assembly 170 (FIG. 10). In operation, the SSDT device 100 is configured to sterilize, store, display and transport one or more (1-4) instrument trays (FIG. 13) in a sealed transparent or translucent container. The SSDT device 100 provides an aseptic interior chamber CH1 after the SSDT device 100 is sterilized in an autoclave.

The CFBC assembly 104, TFL assembly 170 and shelf assembly 150 are described in more detail below. One or more elements of the CFBC assembly 104, TFL assembly 170 and shelf assembly 150 described herein may be omitted or substituted. Furthermore, elements may be added

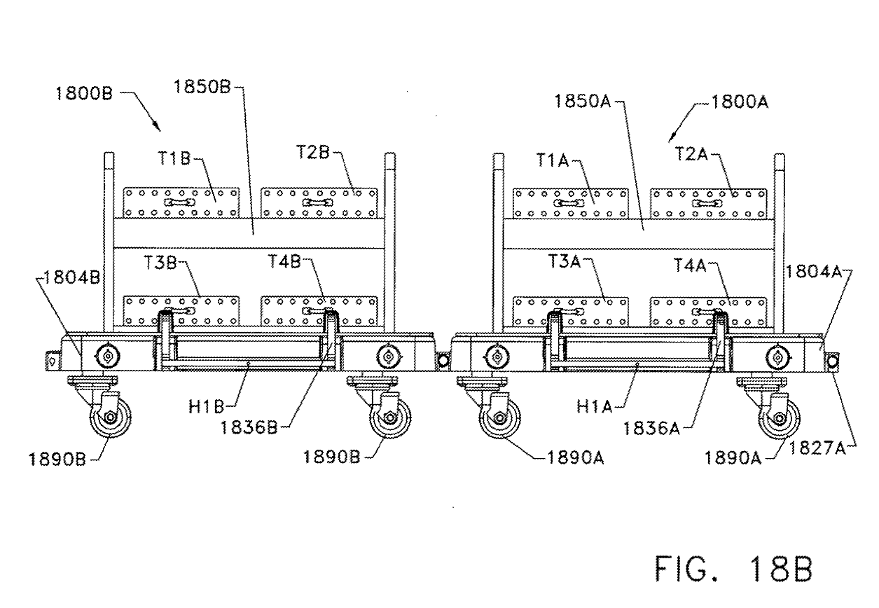

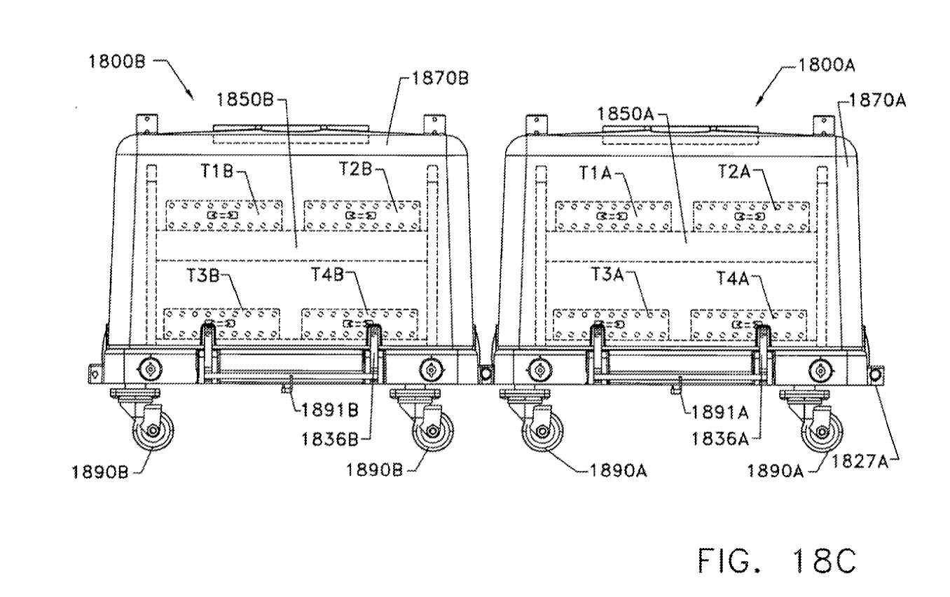

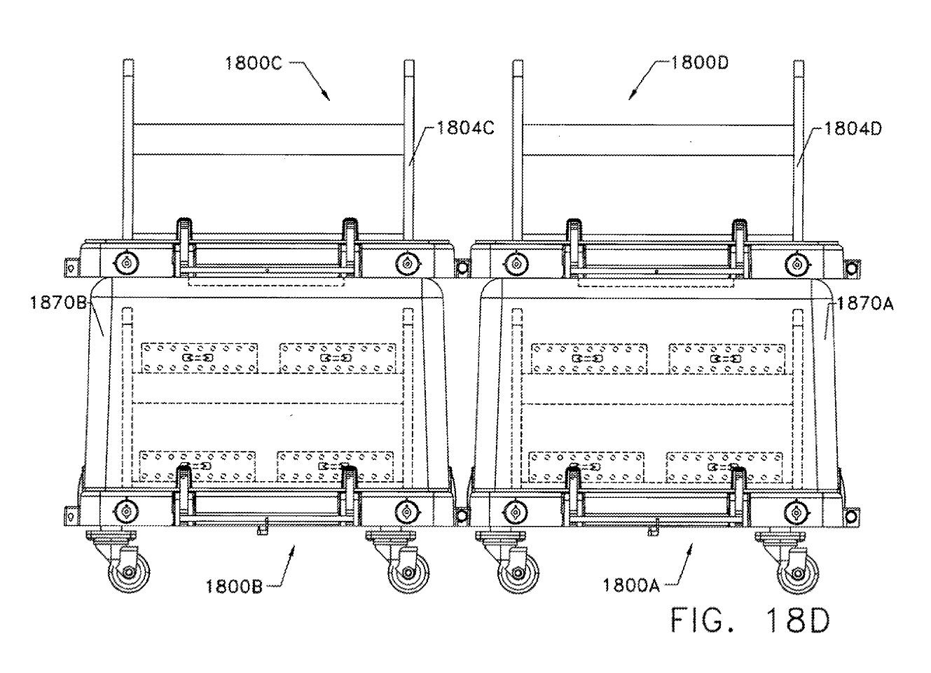

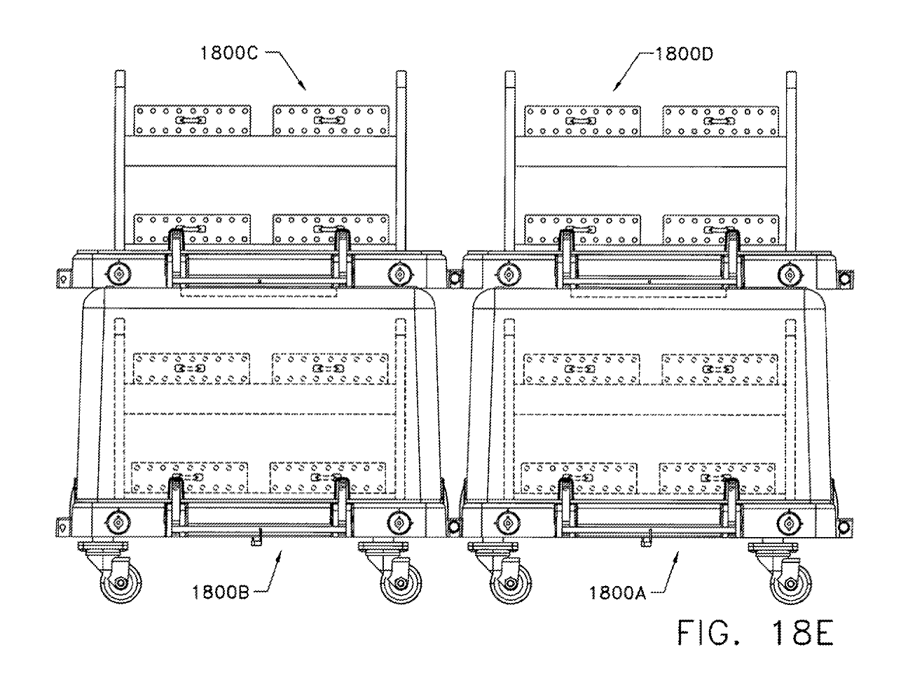

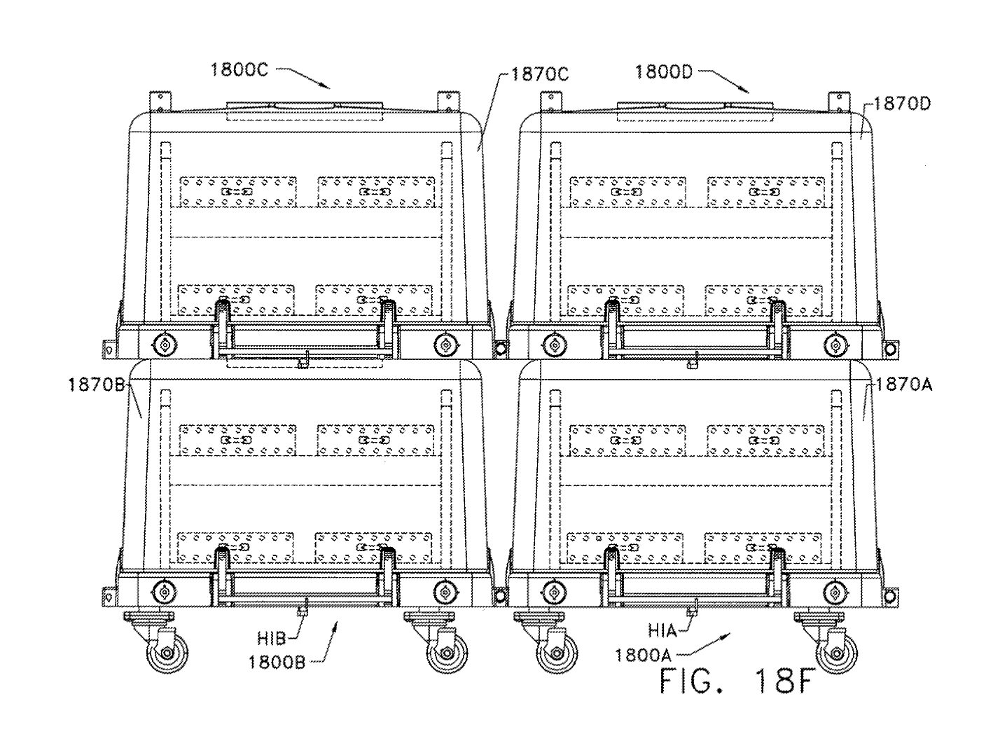

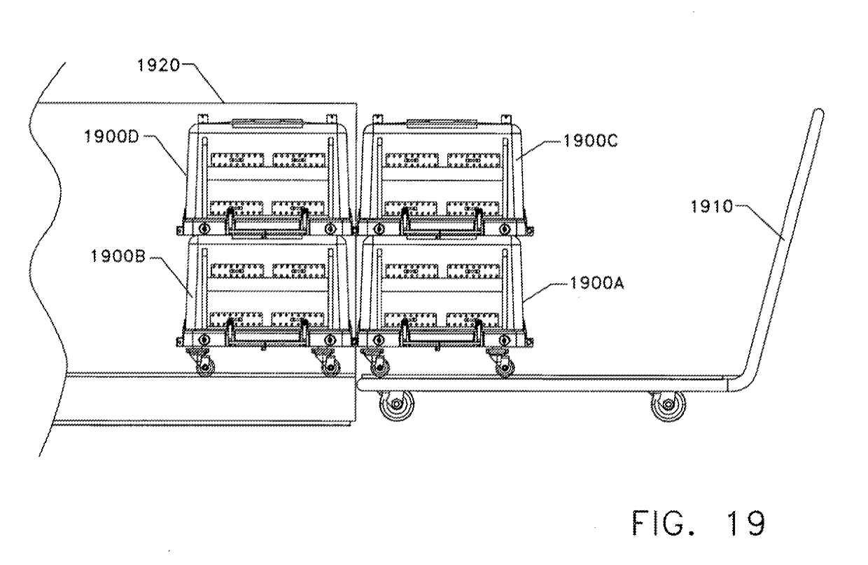

The CFBC assembly 104 includes a base carriage platform 106 that latches and seals a bottom edge of the TFL assembly 170 so that the interior chamber CH1 is closed. The SSDT device 100 is constructed and arranged to provide a user with a configurable, re-configurable and stackable unit so that several SSDT devices may be used together, linked together and moved in unison when linked, as will be described in relation to FIGS. 18A-18F and 19. The SSDT device 100 may also be used individually. When the SSDT device 100 is used individually, ends of the base carriage platform 106 include handle bars 109 and 111. However, the handle bars 109 and 111 double as a hitch or linking mechanisms so that the SSDT device 100 can be easily hitched to adjacent SSDT devices, such as for horizontal stacking and/or moving two or more SSDT devices simultaneously and in unison, as will be described in more detail later.

The CFBC assembly 104 has a length of approximately 22-29.4 inches measured from end to end; a depth of approximately 20-25.5 inches; and a height of approximately 2-4 inches without wheel assemblies. As can be appreciated, the CFBC assembly 104 may have other dimensions but is generally limited to the area within an autoclave.

Each SSDT device 100 includes a shelf assembly 150 which provides a shelf 154 to stack a plurality of instrument trays within the interior chamber under the transparent filtered lid (TFL) assembly 170. As can be appreciated, one or more instrument trays may be placed on top of the shelf 154. The base carriage platform 106 can support one or more instrument trays below the shelf 154. Nonetheless, other objects or items may be placed without the need for a tray above or below the shelf 154 to allow a multitude of different surgical items, products or instruments of varying sizes to be sterilized in a transparent or translucent container. Furthermore, the shelf assembly 150 is removable in order to tit larger objects within the interior chamber defined within the five walls of the TFL assembly 170.

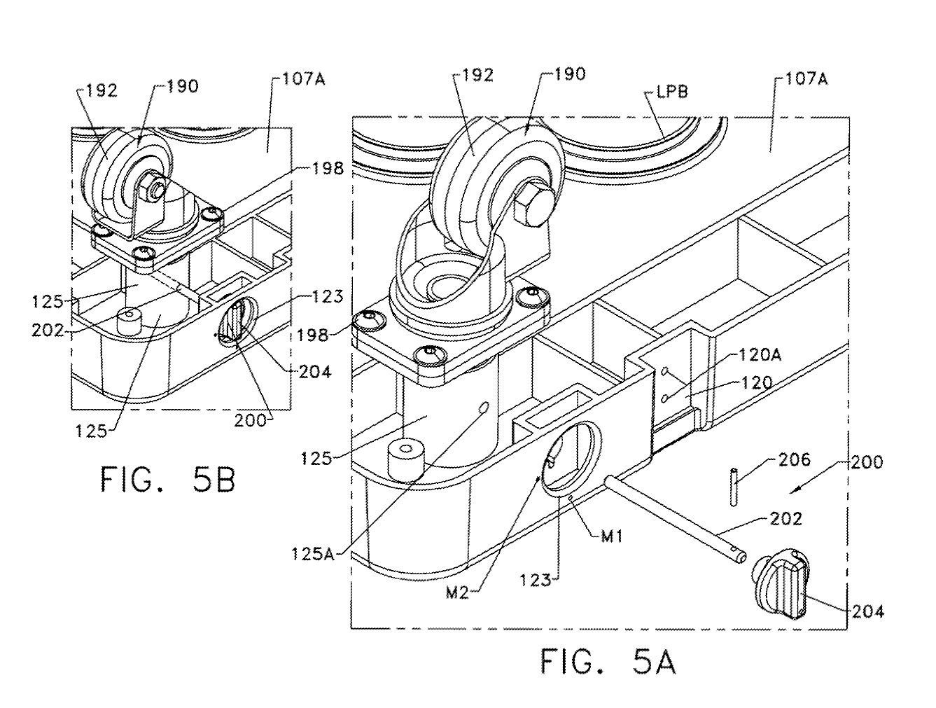

Referring also to FIG. 3, a partial exploded view of the configurable filtered base carriage (CFBC) assembly 104 without wheel assemblies and from a first perspective in accordance with some exemplary embodiments of the present invention is shown. The first perspective is in a direction as viewed from a top side of the CFBC assembly 104. The CFBC assembly 104 includes a base carriage platform 106 to which a plurality of wheel assembles 190 may be selectively attached, as best seen in FIGS. 4 and 5A-5B; a set of latches 132; a second set of latches 136; a gasket or seal 138; and a removable filter assembly 140B installed to cover a vent opening or port 110 for ingress and egress of steam, a sterilant agent and/or heated air.

The removable filter assembly 140B includes a filter keeper frame 141B, a filter 145B and a plurality of fasteners 146B. Each fastener 146B includes a knob head 147A and threaded shank 147B wherein one end of the threaded shank 147B is coupled to the knob head 147A. The filter 145B may be a Hepa Filter to block and filter bacteria and microbial organism from entering into the interior chamber CH1 through the vent opening or port 110. In the exemplary embodiment, the filter 145B may include a means for changing color and/or threads arranged in a pattern to provide a visual indication to indicate that sterilization in accordance with industry standards has been performed.

In the exemplary embodiment, the vent opening or port 110 has an area which is much smaller than the area of the base carriage platform 106. Therefore, the amount of filter material being disposed of after each surgery is relatively small in comparison to the size of the filter sheets used to completely wrap instrument trays. In the exemplary embodiment, the vent opening or port 110 is an opening with no cross supports, grating or other structures within the opening. In an alternate embodiment, grating, cross supports or other structures may be placed to span across the opening.

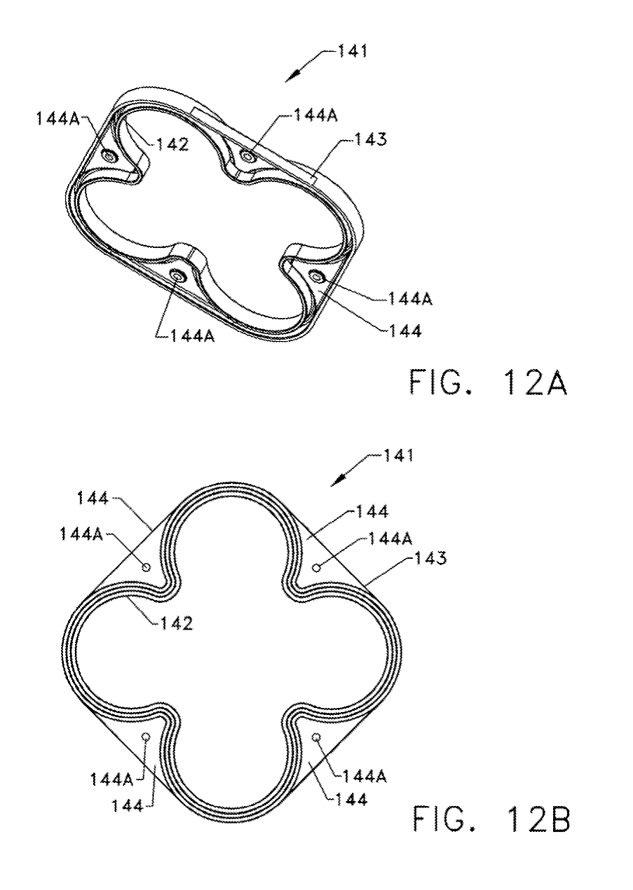

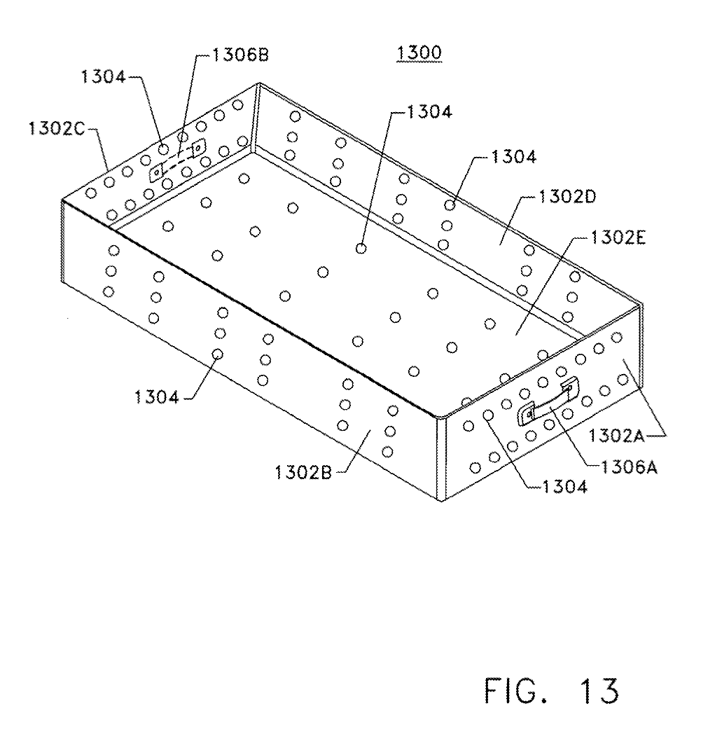

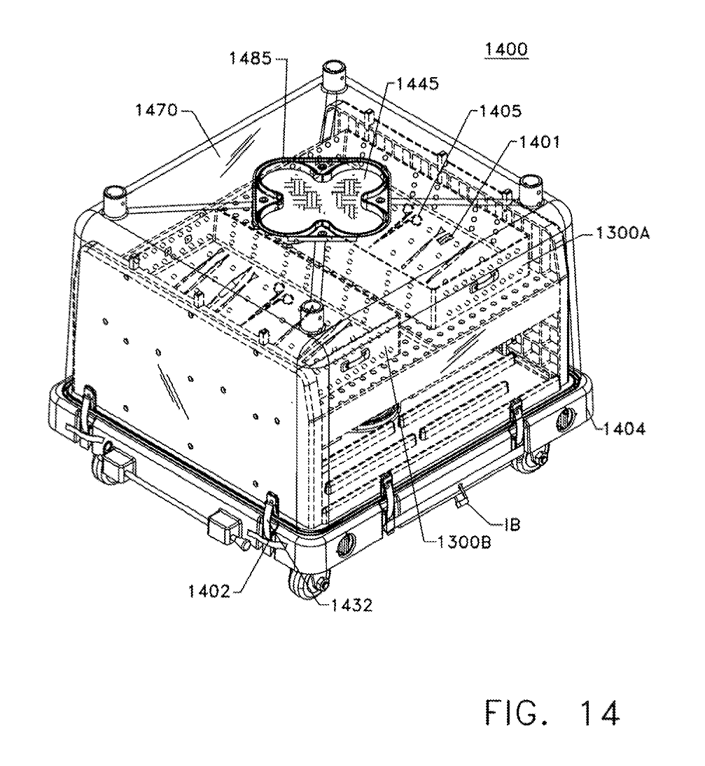

FIGS. 12A and 12B illustrate first and second perspective views of the filter keeper frame 141 in accordance with some exemplary embodiments of the present invention. The filter keeper frame 141 includes an inner flange 142, an outer frame member 143 and pads 144 wherein each pad 144 includes an aperture 144A through which a threaded shank 147B (FIG. 3) of a respective one fastener 146 is received.

In the exemplary embodiment, the filter keeper frame 141 has an interior profile of a four leaf clover. Nonetheless other geometrical and non-geometrical shapes may be used such as, without limitation, circular, elliptical, square, triangular, rectangular, and other shapes. The filter keeper frame 141 is essentially the same as keeper frame 141B in the base carriage platform and keeper frame 141L in the TFL assembly 170.

Returning again to FIGS. 1-3, the base carriage platform 106 includes a horizontal surface 107A and a vertically dependent skirt 107B depending from the horizontal surface 107A. The vertically dependent skirt 107B is outside of the interior chamber CH1 and, specifically, depends from an outer most perimeter edge of the horizontal surface 107A. The horizontal surface 107A has a perimeter seal channel 108 configured to receive therein the gasket or seal 138. The vent opening or port 110 in the horizontal surface 107A includes a frame flange channel 112 dimensioned to receive therein the inner flange 142B to crimp and secure a perimeter of the filter 145B across the vent opening or port 110. The filter 145B may also be stretched taut across the vent opening or port 110. In the exemplary embodiment, the frame flange channel 112 is contoured to track the shape or geometry of the inner flange 142B.

In the exemplary embodiment, when the filter 145 is crimped and secured across the vent opening or port 110, the perimeter edges of the filter 145B may extend slightly beyond or hang over the filter keeper frame 141B.

The horizontal surface 107A includes apertures 113 to be aligned with apertures 144A of the filter keeper frame 141B. The depth of the apertures 113 do not pierce or completely extend through the thickness of the horizontal surface 107A. In other words, the aperture is sufficiently deep to receive and secure the threaded shank 146B of fastener 146 without the threaded shank 146B passing through to the underside of the horizontal surface 107A.

The horizontal surface 107A further includes a plurality of spaced apart treads 114 arranged on the top (interior) side of the horizontal surface 107A and around the vent opening or port 110. The treads 114 are arranged within the interior boundary of the perimeter seal channel 108 and do not obstruct the closing of the TFL assembly 170. The treads 114 are spaced from each other to form a gap to permit steam, sterilant agent and/or heated air flow between and around the treads 114, as well as, provide for fluid run-off during and after a sterilization cycle.

In the exemplary embodiment, the treads 114 are linear raised strip structures and may have varying horizontal lengths. The treads 114 may be irregular in length instead of being linear. For example, the treads 114 may be arranged at an angle or at a slant. Alternately, the treads may be wavy or curved. The treads 114 may all have the same vertical height so that the trays or other objects may be balanced on the top of the treads 114. When a tray or other object is placed on the treads 114, the instrument tray and any aperture of the tray allow steam, sterilant agent or heated air to pass down to the space or gap between treads 114 and out through the vent opening or port 110. Likewise, if necessary, air, steam and a sterilant agent if entering through the vent opening or port 110 would flow within the gaps and up to and through the instrument trays.

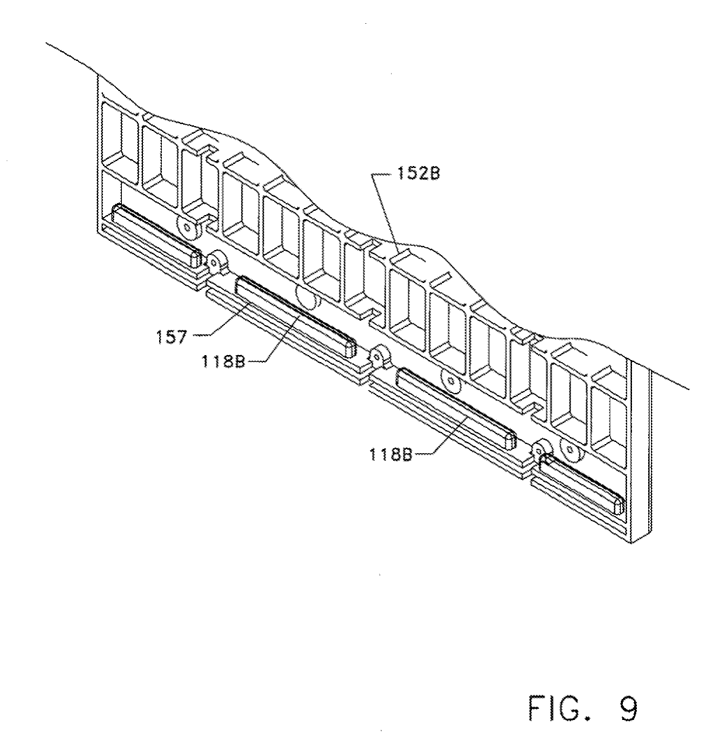

The horizontal surface 107A of the base carriage platform 106 includes one or more base-to-wall connectors 118A adjacent a first interior side of the perimeter seal channel 108 and one or more base-to-wall connectors 118B adjacent to. a second interior side of the perimeter seal channel 108. As will be described in more detail in relation to FIG. 9, the side walls 152A and 152B of the shelf assembly 150 are connected or mounted to the base-to-wall connectors 118A and 118B, respectively.

The one or more base-to-wall connectors 118A include four base-to-wall connectors 118A which are spaced apart. The details of the base-to-wall connectors 118A will be described in more detail in relation to FIG. 9. The gap between adjacent base-to-wall connectors 118A includes an aperture 119. The CFBC assembly 104 further includes optional washer strips 129A and 129B to be described in more detail in relation to FIG. 4. The aperture 119 and washer strips 129A and 129B are optional and may be eliminated. The washer strips 129A and 129B are configured to receive fasteners through aperture 119.

Referring also to FIG. 4, a partial exploded view of the configurable filtered base carriage (CFBC) assembly 104 with wheel assemblies and from a second perspective in accordance with some exemplary embodiments of the present invention is shown. The second perspective is in a direction as viewed from a bottom side of the CFBC assembly 104 and rotated approximately 90 degrees with respect to the view of FIG. 3. The second perspective is taken from an underside of the base carriage platform 106. The vertically dependent skirt 107B includes a plurality of recesses 120 each having at least one aperture 121A formed therein and a plurality of wheel lock receptacles 123. The plurality of wheel lock receptacles 123 will be described in more detail in relation to FIGS. 5A and 5B.

The underside of the horizontal surface 107A includes a plurality of multi-mode base interconnectors 125. The plurality of wheel lock receptacles 123 are in proximity to the multi-mode base interconnectors 125. Markers M1 and M2, as best seen in FIG. 5A, are provided around receptacles 123 on the skirt 107B to indicate an unlocked position and a locked position.

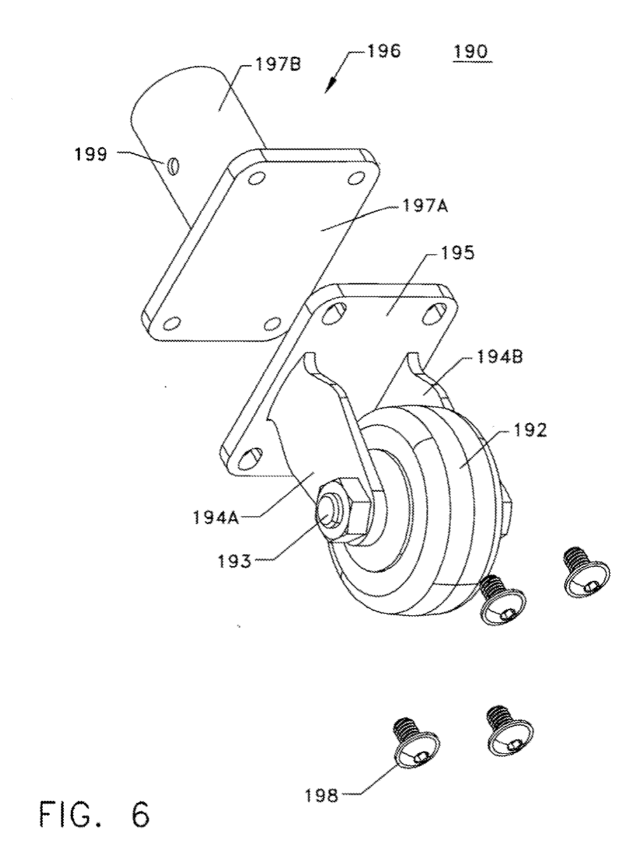

In the exemplary embodiment, the plurality of multi-mode base interconnectors 125 are coupled to the exterior side of the horizontal surface 107A, and in proximity to a corner on an interior side of the vertically dependent skirt 107B. The plurality of multi-mode interconnectors 125 are coupled to, supported by and integrated with the underside of the horizontal surface 107A. The multi-mode interconnectors 125 are isolated from the interior chamber CH1. In a first mode, each multi-mode base interconnector 125 is configured to removably couple to a wheel-to-carriage adaptor 196 of a respective one wheel assembly 190 (FIG. 6). In a second mode, each multi-mode base interconnector 125 is configured to removably couple to a lid interconnector 178. In a third mode, each multi-mode base interconnector 125 is configured to have nothing interconnected thereto and may provide a leg structure when the SSDT device 100 is placed on a support surface.

The underside of the horizontal surface 107A in proximity to the vent opening or port 110 and specifically surrounding the vent opening or port 110, there is a lip LPB for selective attachment of a removable filter cover 139. The removable-filter cover 139 is attached to the lip LPB in a manner similar to the coupling between a plastic lid to a bowl rim. For example, the removable filter cover 139 is snapped on or held in place by a tight fit between the surface of the lip LPB and the cover 139 fitted over the lip. The removable filter cover 139 protects the filter and, after sterilization, provides a strong and durable barrier to prevent bacterial or microbial organism intrusion. The removable filter cover 139 may be made of a transparent plastic or an opaque plastic.

The vertically dependent skirt 107B of the CFBC assembly 104 further includes an aperture or hole 126 formed between two adjacent recesses 120 and provides a means of attaching an integrity band IB (FIG. 14). In the exemplary embodiment, the vertically dependent skirt 107B has a first set of two parallel side walls and a second set of two parallel side walls. The each side wall of the first set of two parallel sides includes a pair of recesses 120 between which the aperture or hole 126 is formed in the side wall.

A first side wall of the second set of two parallel side walls includes a second pair of recesses 120 between which a first set of interlock connectors 127A is coupled, formed or integrated with the first side wall. The first set of interlock connectors 127A includes a first pair of blocks BA1 and BA2 with aligned through holes form therein to receive the handle bar 111. The handle bar 111 is an elongated rod or bar having a flared end 111A and a pin receiving hole 111B in proximity to an end opposite the flared end 111A. The pin receiving hole 111B is configured to have selectively attach thereto a keeper pin P1.

The handle bar 111 is removable such as by removing the keeper pin P1 and sliding the elongated bar of the handle bar 111 out of the first pair of blocks. In the exemplary embodiment, the spacing between the first pair of blocks BA1 and BA2 allows a user's hand to be received therebetween and grasp the handle bar 111.

A second side wall of the second set of two parallel side walls includes a second pair of recesses 120 between which a second interlock connector 127B is coupled, formed or integrated with the second side wall. The second interlock connector 127B includes a block BB1 (FIG. 4) with aligned through holes formed therein being configured to serve as handle bar 109 and also receive another handle bar (e.g., handle bar 111). In the exemplary embodiment, the block BB1 is constructed and arranged as handle bar 109 to be grasped by a user without the need for handle bar 111. Nonetheless, handle bar 111 may also be received in the through holes in block BB1.

The handle bar 111 is removable such as by removing the keeper pin P1 and sliding the elongated bar of the handle bar 111 out of position. The flared end 111A of the handle bar 111 may be removable such as by a threaded connection. The end opposite the flared end 111A may also include a removable flared end. In such a configuration, the keeper pin P1 may be eliminated. Nonetheless, other means of removing and securing the handle bar 111 may be used.

In the exemplary embodiment, the length of the block BB1 is smaller than the spacing between the first pair of blocks BA1 and BA2. The length of block BB1 fits between the blocks BB1 and BB2 and may still allow a user's hand to grasp the handle bar 109. The spacing between the second pair of blocks and the spacing between the first pair of blocks is different. In operation, two adjacent SSDT devices 100 can be hitched or linked together with a single handle bar 111 linking block BB1 to blocks BA1 and BA2. The handle bar 111 can be slid into place and the keeper pin P1 installed to hitch two adjacent SSDT devices together.

The CFBC assembly 104 further includes a carriage support assembly 133 having a set of washer strips 129C and 129D to be fastened to washer strips 129A and 129B (FIG. 3). The washer strips 129C and 129D are further configured to be attached to the support grid 134 via a plurality of fasteners 134A. Fasteners 134A are also coupled to the washer strips 129A and 129B. It should be recognized that the washer strips 129C and 129D are optional. Furthermore, the support grid 134 may be attached to the base carriage platform 106 via the vertically dependent skirt 107B or other structures independent of the horizontal surface 107A.

FIG. 6 illustrates an exploded view of a wheel assembly 190 in accordance with some exemplary embodiments of the present invention. The wheel assembly 190 includes a caster wheel 192 having a central axle 193. Opposite ends of the central axle 193 have coupled thereto one end of support arms 194A and 194B. The other end of the support arms 194A and 194B are coupled to an adaptor connector plate 195.

The wheel assembly 190 further includes a wheel-to-carriage adaptor 196. The adaptor 196 includes a connector plate 197A and a wheel-to-carriage interconnector 197B. The connector plate 197A is fastened to the adaptor connector plate 195 via fasteners 198. The wheel-to-carriage interconnector 197B includes a through hole 199 to be described in more detail below.

FIGS. 5A and 5B illustrate an unlocked state and locked state of a wheel assembly 190 when attached to the base carriage platform 106 in accordance with some exemplary embodiments of the present invention. The wheel-to-carriage interconnector 197B is configured to be coupled to the multi-mode base interconnector 125. The wheel-to-carriage interconnector 197B is configured to be locked to the multi-mode base interconnector 125 via the wheel locking pin 200. The hole 199 is configured to be aligned with hole 125A formed in the multi-mode base interconnector 125.

The wheel locking pin 200 includes, in general, an elongated shank 202 configured to have a locking knob 204 coupled or attached thereto via a pin 206. The elongated shank 202 is received in the hole 125A and hole 199 and passes therethrough. The locking knob 204 is received in the wheel lock receptacles 123 and is configured to be turned approximately 90 degrees from marker M1 to marker M2. At marker M2, the wheel assembly 190 is locked. Other locking arrangements may be used.

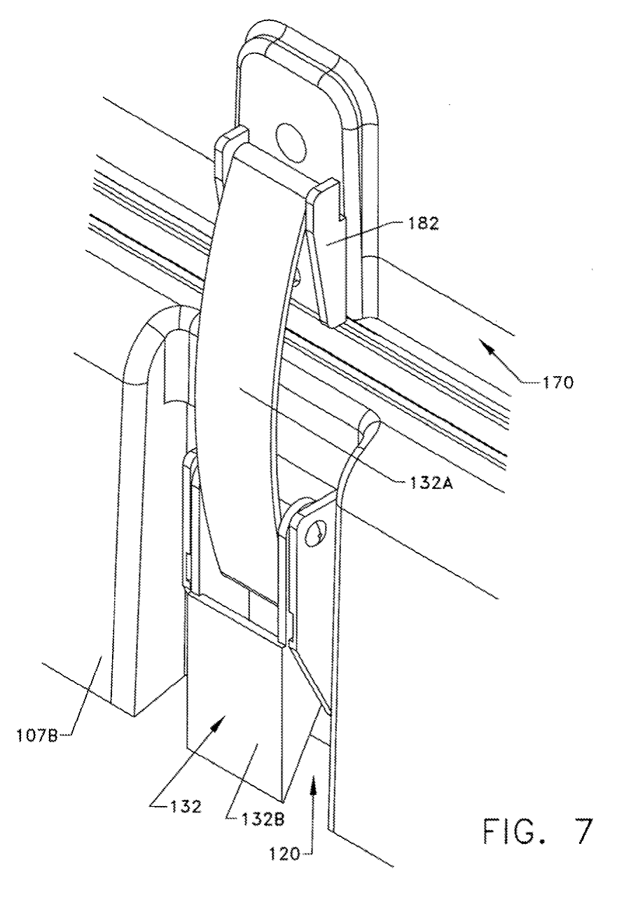

FIG. 7 illustrates a latch 132 to latch the transparent filtered lid (TFL) assembly 170 in accordance with some exemplary embodiments of the present invention. The latch 132 includes a latching arm 132A configured to be engaged to and disengaged from a latch locking bracket 182 on the TFL assembly 170. The latch 132 is configured to be attached in recess 120. The latch 132 includes a latch actuator 132B, pivotally coupled to the latching arm 132A. Latches are well known in the art and no further discussion of the operation of the latch is necessary. The latch actuator 132B is generally protected within recess 120.

In operation, the latch 132 can be taped over with sterilization tape, as best seen in FIG. 14.

The latches 132 are independently operated. The latches 136 are similar to latches 132 except that the latch actuator 132B of two side by side latches 136 are linked together via a latching bar 136A. The latching bar 136A includes a hole H1 to be aligned with hole 126. An integrity band IB can be journalled through hole H1 and hole 126 locked in a manner similar to a pad lock. The integrity band IB (FIG. 14) is a plastic lock or tie commonly used in the medical industry and suitable for autoclave environments.

As can be appreciated, the two latches 136 may be modified with a single latch or may include more than two latches 136. However, the latch or latches 136 should be banded. Furthermore, latches 132 may be banded. The SSDT device may include one or more latches which are configured to be banded with an integrity band.

FIG. 8 illustrates an exploded view of a shelf assembly 150 in accordance with some exemplary embodiments of the present invention. The CFBC assembly 104 supports thereon a shelf assembly 150. The shelf assembly 150 includes a pair of parallel side walls 152A and 152B removable coupled to the CFBC assembly 104, as best seen in FIG. 3. The shelf assembly 150 further includes a shelf 154 configured to be supported horizontally and in parallel with the CFBC assembly 104. The shelf 154 includes a plurality of holes 156 formed therein to permit the ingress and/or egress of steam, air or a sterilant agent, as needed.

The shelf 154 includes at least one shelf connector 158SA and 158SB configured to interconnect the shelf 154 to the side walls 152A and 152B, respectively. Thus, the side walls 152A and 152B include at least one wall connector (only 158WB shown). Fasteners 159 (e.g., screws, bolts) are provided to further fasten the at least one shelf connector 158SA and 158SB to the at least one wall connector (only 158WB shown).

In the exemplary embodiment, the at least one shelf connector 158SA and 158SB are male connectors while the at least one wall connector are female connectors. Nonetheless, the at least one shelf connector 158SA and 158SB may be female connectors while the at least one wall connector would be male connectors. The connectors between the shelf and the side walls 152A and 152B may be snapped together or friction tit coupled. Nonetheless, the shelf and sidewalls may be integrated or coupled into a single structure requiring no fasteners. The fasteners may be screws, bolts, or other fasteners.

In the exemplary embodiment, one or more reinforcement vertical rails 160A and 160B are configured to be coupled to the side walls 152A and 152B, respectively, and/or the base carriage platform 106. Additionally, one or more shelf reinforcement horizontal rails 162 are configured to be coupled to an underside of the shelf 154. The shelf reinforcement horizontal rails 162 are essentially parallel to the base carriage platform 106 and perpendicular to the side walls 152A and 152B. The vertical rail 160A and 160B are essentially parallel and aligned.

The rails 160A and 160B and rails 162 may be made of a non-corrosive metal such as stainless steel or other metals.

The side walls 152A and 152B include channels 161A and 161B, respectively to receive and install the rails 160A and 160B, respectively. The channels 161A and 161B extend the length of the side walls 152A and 152B. The side walls 152A and 152B further include apertures A1 for fasteners 159 to fasten the one or more shelf reinforcement rails 162 and rails 160A and 160B thereto.

The side walls 152A and 152B and/or shelf 154 may be made of clear polypropylene, a clear plastomer, propylene homopolymers or other transparent or translucent plastics.

FIG. 9 illustrates a partial view of a shelf wall 152B and the base-to-wall connectors 118A of the base carriage platform in accordance with some exemplary embodiments of the present invention. The base carriage platform 106 (FIG. 3) includes one or more base-to-wall connectors 118A on one side thereof. Additionally, side wall 152B includes one or more bottom wall connectors 157. The bottom wall connectors 157 are female connectors while the base-to-wall connectors 118A are male connectors. In operation, the shelf wall assembly 150 is removable from the base carriage platform. The base-to-wall connectors 118A allow the shelf wall 152B to be removably mounted to the base carriage platform 106 (FIG. 3) by lifting the shelf wall 152B above the height of the base-to-wall connectors 118A. As can be appreciated other connections or connectors may be used.

FIG. 10 illustrates an exploded view of the transparent filtered lid (TFL) assembly 170 in accordance with some exemplary embodiments of the present invention. The TFL assembly 170 includes a transparent box-shaped structure 172 having in general four vertical walls 174A, 1748, 174C and 174D and a top wall 174E. The area confined within the four vertical walls 174A, 174B, 174C and 174D and a top wall 174E is hollow and forms the interior chamber CH1. The four vertical walls 174A, 174B, 174C and 174D has a bottom rim or edge 176 configured to be sealed to the base carriage platform 106 of the CFBC assembly 104 via gasket or seal 138.

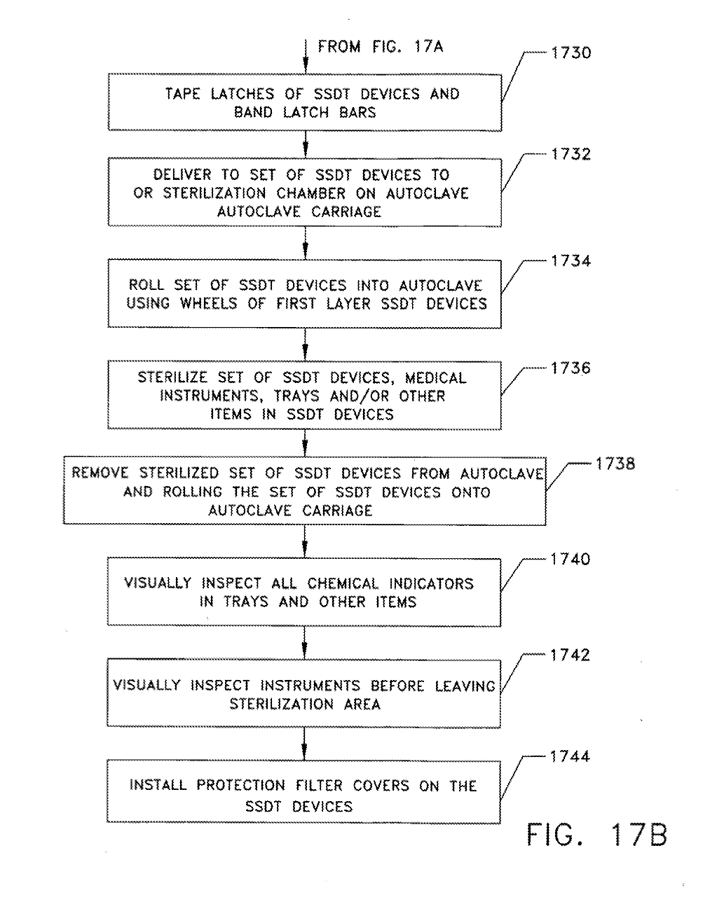

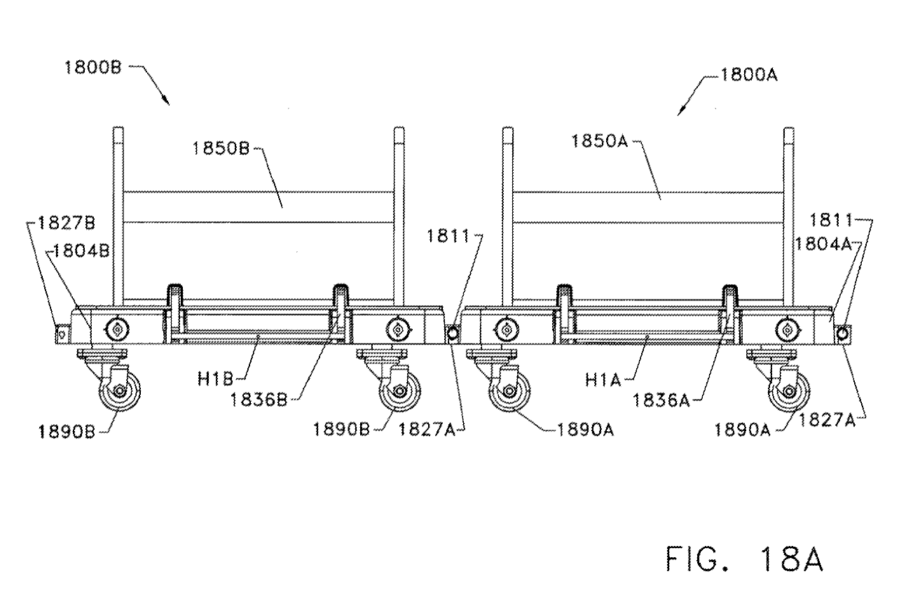

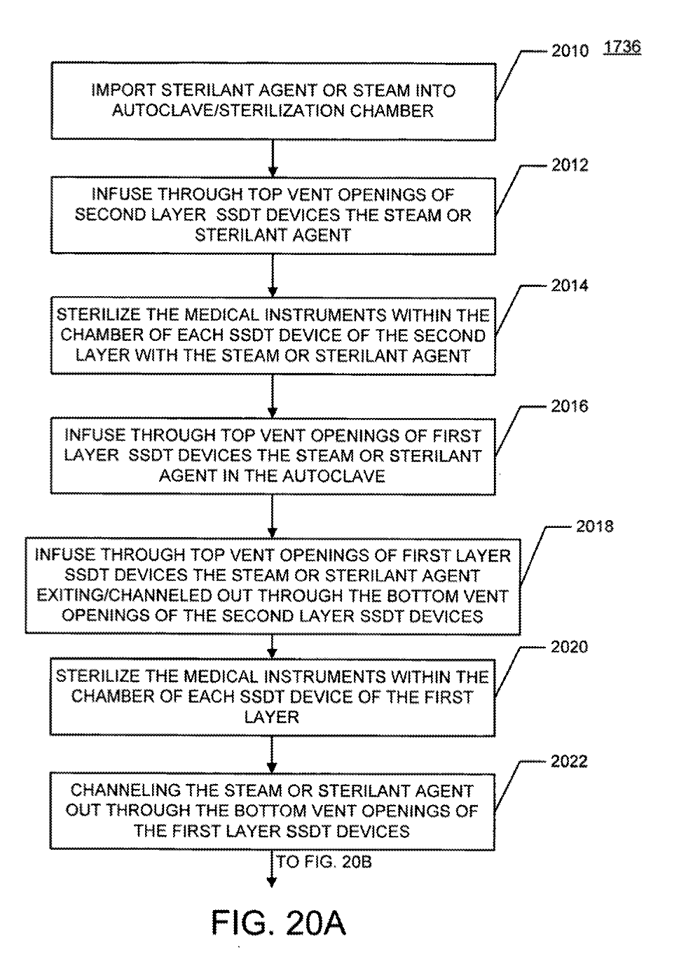

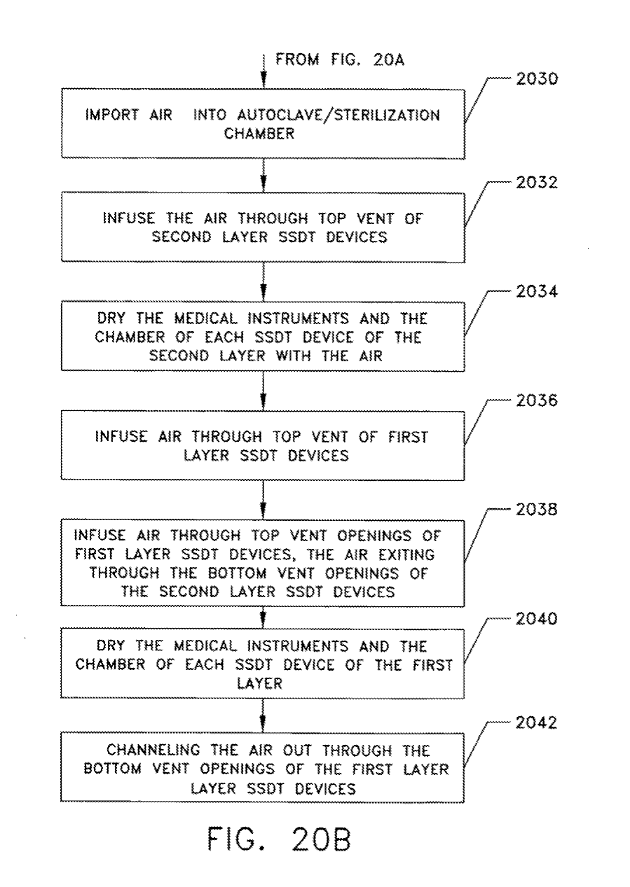

The top wall 174E is sloped toward the four vertical walls 174A, 174B, 174C and 174D to permit run-off of any fluid adhering to wall 174E as the result of the sterilization cycle, described in detail below in relation to FIGS. 17B or 20A and 20B. Each of the vertical walls 174A, 174B, 174C and 174D includes a pair of latch connecting pads 180. Each latch connecting pad 180 includes apertures 180A. The apertures 180A do not pierce or extend completely through the vertical walls 174A, 174B, 174C and 174D so that bacteria or other microorganisms may enter the interior chamber CH1.

Each latch connecting pad 180 is constructed and arranged to have attached thereto a latch locking bracket 182 via fasteners 183. The latches 132 and 136 when latched to locking bracket 182 pulls or draws the edge 176 down into the gasket or seal 138.

The removable filter assembly 140L includes a filter keeper frame 141L, a filter 145L and a plurality of fasteners 146L. Each fastener 146L includes a knob head 146A and threaded shank 146B wherein one end of the threaded shank 146E is coupled to the knob head 146A. The filter 145L may be a Hepa Filter.

In the exemplary embodiment, the filter keeper frame 141L has an interior profile of a four leaf clover. Nonetheless other geometrical and non-geometrical shapes may be used such as, without limitation, circular, elliptical, square, triangular, rectangular, and other shapes. Although the removable filter assemblies 140L and 140B have the same general shape, the shapes of the removable filter assembly 140L may be different and varied from the removable filter assembly 140B.

A filter cover 139 is also used to cover and protect the filter 145L. The filter cover 139 is intended to be attached to the top exterior side of the transparent box-shaped structure 172.

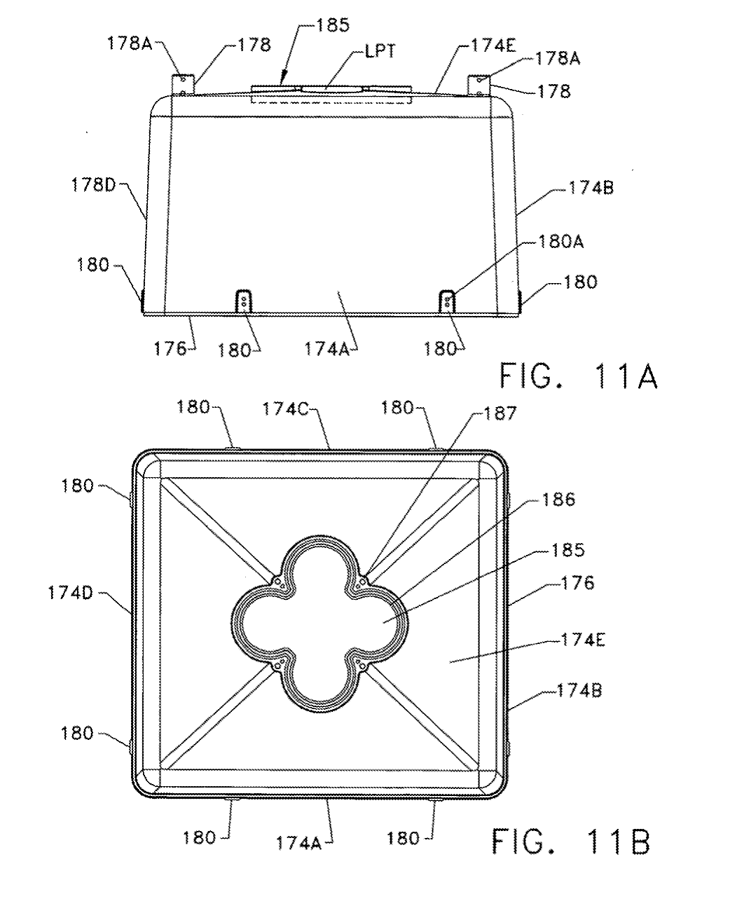

FIGS. 11A and 11B illustrate side and bottom views of the transparent box-shaped structure 172 in accordance with some exemplary embodiments of the present invention. The TFL assembly 170 includes a top vent opening or port 185 formed in the top wall section 174E. The vent opening or port 185 is configured to have a removable filter assembly 140L coupled thereto from the underside of top wall section 174E. The removable filter assembly 140L is essentially the same as filter assembly 140B, as previously described in relation to FIGS. 3 and 12A-12B.

The top exterior surface of the top wall 174E in proximity to the corners includes a set of lid interconnectors 178 having at least one aperture 178A formed therein. The set of lid interconnectors 178 allows two adjacent SSDT devices to be horizontally stacked vertically wherein the CFBC assembly 104 of one SSDT device can be stacked above the other SSDT device as described in relation to FIGS. 18D-18F and 19.

The top vent opening or port 185 includes a frame flange channel 186 in an underside of the top wall 174E, the frame flange channel 186 being dimensioned to receive therein the inner flange 142L to crimp and secure a perimeter of the filter 145L across the vent opening or port 185. The filter 145L may also be stretched taut across the vent opening or port 185. A lip LPT is coupled to the top of wall 174E wherein the filter cover 139 is selectively attached to lip LPT to cover the filter 145L.

The top vent opening or port includes apertures 187 in the underside of the top wall 174E. The apertures 187 are to be aligned with apertures of the filter keeper frame 141L. The depth of the apertures 187 does not extend through the thickness of the top wall 174E. In other words, the aperture 187 is sufficiently deep to receive and secure the threaded shank 146B of fastener 146L without the threaded shank 146B passing through to the underside of the top wall 174E. The apertures should be concealed or closed to the exterior and do not provide a path for bacteria or microbial organism intrusion.

In the exemplary embodiment, the TFL assembly 170 has a length of approximately 20-26 inches, a depth of approximately 18-24 inches and a height of approximately 10-16 inches. The height is measured from the edge 176 to the top of interconnectors 178. As can be appreciated, the TFL assembly 170 may have other dimensions but is generally limited to the area within an autoclave.

In the exemplary embodiment, the transparent box-shaped structure 172 and interconnectors 178 are made of clear polypropylene, a clear plastomer, propylene homopolymers or other transparent or translucent plastics. All transparent material may be translucent.

FIG. 13 illustrates a transparent tray 1300 for placement and sterilization of medical instruments and other items used in surgical procedures in accordance with some exemplary embodiments of the present invention. In an exemplary embodiment, the tray 1300 includes four vertical walls 1302A, 1302B, 1302C and 1302D and a bottom horizontal wall 1302E. Each wall 1302A, 1302B, 1302C, 1302D and 1302E has a plurality of holes 1304 formed therein to permit the passage of steam, a sterilant agent and/or air. The holes 1304 allow fluids if present to drip through such holes. The tray 1300 further includes tray handles 1306A and 1306B on each end walls 1302A and 1302C, respectively. In FIG. 13, handle 1306B is shown in phantom. 1001031 The four vertical walls 1302A, 1302B, 1302C and 1302D and a bottom horizontal wall 1302E of tray 1300 are transparent or translucent and permits visual inspection of a chemical indicator CI (FIG. 14) or other indicators as well as the instruments from a variety of angles.

The tray 1300 is made of clear polypropylene, a clear plastomer, propylene homopolymers or other transparent or translucent plastics. In an alternate embodiment, the tray 1300 may be a stainless steel tray. The tray 1300 may be made of other materials (plastic or metals) suitable for repeated use in a high heat and wet environments of an autoclave.

The tray 1300 has a generally rectangular shape. However other shapes may be used. For example, tray 1300 may be square or round.

FIG. 14 illustrates a loaded sterilization, storage, display and transportation (SSDT) device 1400 for use in the transparent sterilization, storage and transportation system in accordance some exemplary embodiments of the present invention. The SSDT device 1400 is essentially the same as SSDT device 100. Therefore, only the differences will be described.

In FIG. 14, the SSDT device 1400 is shown loaded with two trays 1300A and 1300B, wherein the trays include a plurality of instruments 1405 to be used in a surgery. In the exemplary embodiment, a chemical indicator (CI) 1401 is placed in the trays. The CI 1401 is viewed through the TFL assembly 1470.

In the exemplary embodiment, no trays are shown on the CFBC assembly 1404. However, trays may be placed on the CFBC assembly 1404. The filter 1445 is shown covering the vent opening or port 1485. The latches 1432 are shown taped with strips of sterilization tape 1402. Additionally, latch bars are banded with integrity bands IB such that the latch bars if lifted would break the integrity band IB to indicate a compromise or tampering.

The transparent properties of the TFL assembly 1470 allow multiple trays to be viewed so that instruments can be quickly identified and accessed. The plurality of trays can be viewed 360 degrees while the TFL assembly 1470 remains sealed and latched.

The SSDT device 1400 has a generally rectangular or box shape. However, other geometrical and non-geometrical shapes may be used.

FIG. 15 illustrates a flowchart of a sterilization method 1500 using a transparent sterilization, storage and transportation system in accordance some exemplary embodiments of the present invention. The sterilization method 1500 includes, in general, three phases. The three phases include a pre-sterilization phase 1510; a sterilization phase 1512 using the transparent sterilization, storage and transportation system as described herein; and a post-sterilization phase 1514. Each phase will be described in detail below.

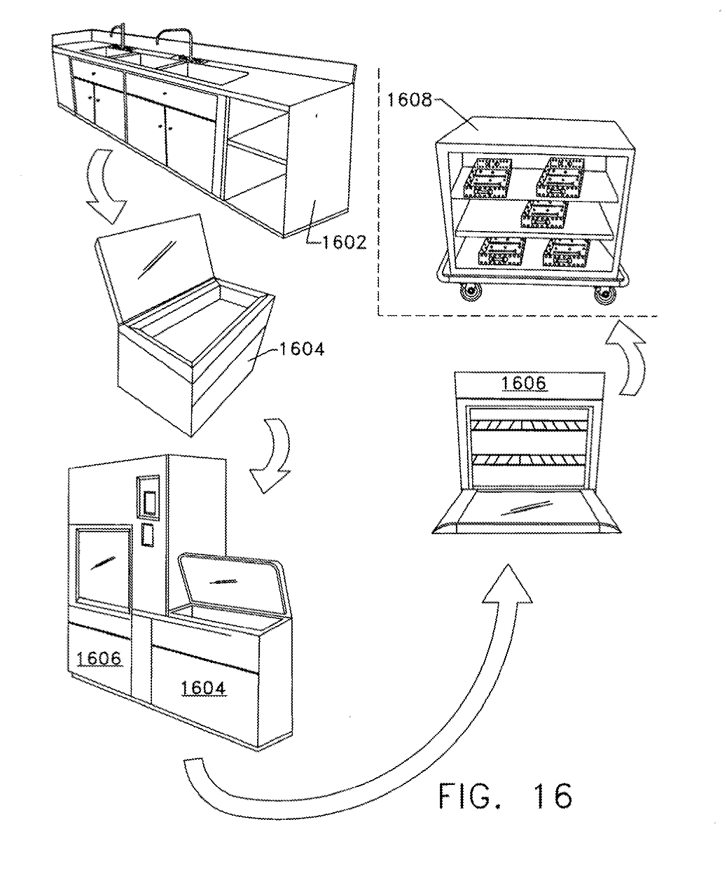

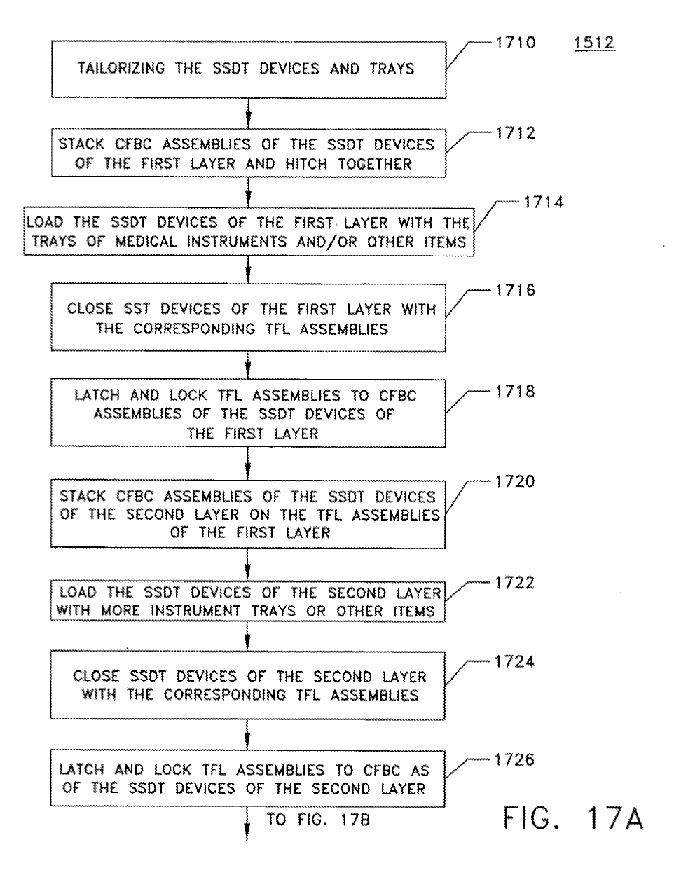

FIG. 16 illustrates stages of the pre-sterilization phase 1510 of the sterilization method 1500 of FIG. 15. Before a medical instrument tray (e.g., 1300 of FIG. 13) is sterilized, the surgical instruments and trays are sanitized and cleaned in a variety of stages. In FIG. 16, the stages include a rinsing stage 1602 which takes place using sinks, a cleaning stage 1604 where the trays and/or instruments may be subjected to a washing cycle, a pre-sterilization washing process, or sonic cleaning process. After stage 1604, the cleaned or washed trays and instruments are passed through at stage 1606 to a sterile area and await preparation for sterilization at stage 1608. The trays and instruments at stage 1608 may be placed on a wheeled cart.

As can be appreciated, one or more of the steps may be omitted in the pre-sterilization phase 1510 of the medical instrument tray sterilization process.

One or more of the steps of the sterilization method 1500 may be performed in the depicted order, contemporaneously with other steps, in parallel or in a different order. FIGS. 17A-17B illustrates a flowchart of the sterilization phase 1512 of the sterilization method 1500 in accordance with the present invention. In the exemplary embodiment, the sterilization phase 1512 of the method 1500 described herein utilizes a translucent sterilization, storage and transportation system, as described in relation to FIGS. 18A-18F and 19. The system may further include transparent or translucent trays 1300 (FIG. 13) or other trays suitable for autoclave environments.

The sterilization phase 1512 includes tailorizing the SSDT devices 1800A-1800D (FIGS. 18A-18F) and trays, such as tray 1300, at Step 1710. The number of SSDT devices may vary. The medical instrument trays are loaded and packaged for a surgical procedure. In many instances, the medical instrument trays are packaged and tailorized based on a surgeon's customized list of medical instruments for the procedure being performed.

At Step 1710, a chemical indicator (CI) 1401 (FIG. 14) is placed in each tray. The CI 1401 should be placed in a tray that permits the CI 1401 to be visually inspected without moving instruments around. Other indicators such as chemical indicators may be used. The CI 1401 is sensitive to or responsive to the temperature changes as the result of sterilization by an autoclave or other sterilization chamber meeting medical industry standards.

Simultaneously with, before or after the packaging of the trays 1300, each SSDT device 1800A, 1800B, 1800C and 1800D of the SSDT system is also tailorized. The SSDT system includes a plurality of SSDT device's 1800A-1800D. The system may include transparent trays 1300 or other metal trays. At a minimum each SSDT device 1800A-1800D has two internal filters (e.g., filters 1458 and 145L) to be installed or replaced to cover vent openings or ports 110 and 185 in the CFBC assembly 106 and the TFL assembly 170, respectively. The filters 145B and 145L are installed on the interior and are generally tamperproof once installed and the TFL assembly 170 latched. When the filters 145B and 145L are installed, the vent openings or ports 110 and 185 are left open but are filtered so that there is ingress and egress of steam, a sterilant agent and/or air through the vent openings or ports when sterilization takes place in the autoclave or sterilization chamber.

Tailorizing the SSDT devices for a particular surgeon may include installing or removing wheel assemblies 190. The SSDT devices 1800A-1800D are modular and may be selectively linked for a particular surgery. Therefore, if the shelf assembly 150 is not necessary, the shelf assembly 150 may be removed. Depending on the size of some items a surgeon may request, the shelf assembly 150 may need to be removed to provide clearance within the interior chamber CH1. Removal of the shelf assembly 150 would provide a larger distance or clearance between the underside of the TFL assembly 170 and the base carriage platform 106 to accommodate objects that may be much larger than a tray.

Furthermore, the shelf assembly may be removed so that the SSDT device would support one or two trays. For example, if a particular surgeon only requires ten (10) trays, three SSDT devices may be used. In one example, one SSDT device would include four trays; a second SSDT device would include four trays; and a third SSDT device would include two trays and/or other items.

If a second surgeon only wanted 4 trays, when sterilizing, three SSDT devices would be associated and tailorized for the first surgeon while a fourth SSDT device would be associated and tailorized for a second surgeon. When autoclaving, one of the top SSDT devices can be removed after sterilization so that it can be separately transported to a separate operating room (OR). The translucent or transparent properties, allows the personnel to easily visually inspect the instruments and separate SSDT devices without compromising the integrity banding, CIs and latch taping.

Referring now to FIGS. 18A-18C, the hitching and loading of a first level SSDT devices 1800A and 1800B of the sterilization, storage, display and transportation (SSDT) system in accordance with some exemplary embodiments of the present invention are shown. At Step 1712, a first layer of SSDT devices 1800A and 1800B are stacked such as on top of an autoclave carriage 1910 (FIG. 19). The stacking includes stacking the CFBC assemblies 1804A and 1804B side-by-side horizontally and hitching or linking the assemblies with handle bar 1811 coupled to interlock connectors 1827A. In FIG. 18A, the CFBC assemblies 1804A and 1804B are hitched together and include wheel assemblies 1890A and 1890B, respectively, so that the CFBC assemblies 1804A and 1804B can be moved in unison and simultaneously. The SSDT devices 1800A and 1800B in FIG. 18A have no lid, thus the shelf assemblies 1850A and 1850B are exposed for loading trays.

In FIG. 18A, the interlock connector 1827A on CFBC assembly 1804A is shown with handle bar 1811 attached. The handle bar 1811 may be used has a handle bar for grasping by hospital personnel such as to pull the SSDT device. The interlock connector 1827B serves as a handle bar (e.g., handle bar 109) and is shown without a handle bar 111. Nonetheless, the handle bar 111 may be attached if preferred. The latches 1836A and 1836B are unlatched. Holes H1A and H1B in the latch bar would be used to connect an integrity band, as will be described in detail later.

The horizontal stacking of the SSDT devices, and/or hitching SSDT devices serve to create a customized set of SSDT devices for use in packaging medical instruments for a single surgical procedure. Each SSDT device 1800A-1800D may be used to sterilize up to four trays 1300 with surgical instruments or other trays. Two SSDT device 1800A and 1800B may be used to sterilize 4-8 instrument trays. Nonetheless, each SSDT device may sterilize one or more instrument trays up to four trays per SSDT device. As can be appreciated, the SSDT device may be made bigger to support additional instrument trays.

Most autoclaves would only fit two horizontally stacked SSDT devices 1800A and 1800B on a first layer. Nonetheless, the number of horizontally stacked SSDT devices is a function of the size of the autoclave. The linking or hitching of the SSDT devices 1800A-1800B allows the set of SSDT devices to be easily moved off and on the autoclave carriage 1910 (FIG. 19).

In the exemplary embodiment, the horizontally stacked SSDT devices are hitched or linked together so that when the set of SSDT devices are off-loaded from the autoclave carriage 1910, all SSDT devices 1800A, 1800B, 1800C and 1800D move together in unison. The wheel assemblies 1890A and 1890B of the first layer SSDT devices 1800A and 1800B are used to roll the set of SSDT devices 1800A, 18008, 1800C and 1800D off and on to the autoclave carriage 1910.

At Step 1714, a plurality of loaded medical instrument trays T1A-T4A are placed in the first SSDT device 1800A and trays T1B-T4B are placed on the second SSDT device 1800B of the first layer, as best seen in FIG. 18B. In the example, two trays are placed on each shelf assembly 1850A and 1850B and two trays below the shelf assemblies.

At Step 1716, the TFL assemblies 1870A and 1870B of the first and second SSDT devices 1800A and 1800B, respectively, are installed to close the SSDT devices 1800A and 1800B. Thereafter, at Step 1718, the TFL assemblies 1870A and 1870B are latched and locked which automatically seals the TFL assemblies 1870A and 1870B to the CFBC assemblies 1804A and 1804B, respectively.

In the exemplary embodiment, the integrity bands 1891A and 1891B are installed to band the latching bar. The integrity band serves to indicate that the latches have not been opened since sterilization. Each SSDT device 1800A and 1800B includes two integrity bands. The integrity band may be installed before Step 1720, during Step 1730 or some other time before the system is placed in the autoclave.

At Step 1720, a third CFBC assembly 1804C of a third SSDT device 1800C and/or a fourth CFBC assembly 1804D of a fourth SSDT device 1800D are stacked vertically on top of the SSDT device 1800B and 1800A, respectively, as best seen in FIG. 18D. In this embodiment, the CFBC assemblies 1804C and 1804D do not include wheel assemblies and are configured to connect to the TFL assemblies of the first layer. At Step 1722, the third SSDT device 1800C and/or 1800D are loaded with one or more trays filled with instrument or other items.

At Step 1724, the TFL assemblies 1870C and 1870D of the third and fourth SSDT devices 1800C and 1800D, respectively, are installed to close the SSDT devices 1800C and 1800D. Thereafter, at Step 1726, the TFL assemblies 1870C and 1870D are latched and locked which automatically seals the TFL assemblies 1870C and 1870D to the CFBC assemblies 1804C and 1804D, respectively, of the SSDT devices 1800C and 1800D, respectively, as best seen in FIG. 18F.

Referring again to FIGS. 18A-18F, the SSDT system is modular and adaptable. When conducting a surgery, the modular properties allow various combinations of SSDT devices to be used in a variety of stacked profiles.

In an exemplary embodiment, all SSDT devices 1800A-1800D include a set of interlock connectors 1827A and 1827B that allow the devices to be selectively horizontally stacked and connected. The interconnectors (e.g., interconnectors 178) on the TFL assemblies allow the devices to be vertically stacked. As can be appreciated, the size of the SSDT device 1800A-1800D may vary. For example, the second layer SSDT devices stacked above the first layer SSDT device may have a shorter height. Nonetheless, all devices may have the same or different height, width and depth.

In an alternate embodiment, the SSDT devices on the second layer may be made smaller so that two side-by-side SSDT devices can fit over a single SSDT device on the first layer. In this case additional interconnectors on the TFL assemblies may be needed.

During sterilization, the system, since modular, may only use three stacked SSDT devices. The three SSDT devices may include a primary SSDT device (e.g., SSDT device 1800A) with one SSDT device 1800D stacked vertically above the SSDT device 1800A and another SSDT device 18008 stacked horizontally with respect to the primary SSDT device 1800A.

Alternately, in lieu of SSDT device 1800D, the SSDT device 1800C could be stacked on top of SSDT device 1800B with location of the SSDT 1800D empty.

Nonetheless, the system may include four SSDT devices 1800A-1800D. The four SSDT devices 1800A-1800D may each be horizontally stacked in series. Alternately, the four SSDT devices 1800A-1800D may be stacked as two rows and two columns. Other arrangements are provided. If only two SSDT devices are needed, the stacking arrangement of SSDT devices 1800A and 1800D may be used.

Referring now to FIG. 17B, at Step 1730, integrity band(s) are attached to at least one latching bar of each SSDT device 1800A-1800D; and optionally one or more latches 132 without the latching bar may be taped with sterilization tape, as shown in FIG. 14. In the exemplary embodiment, integrity bands are attached to parallel latching bars on opposite sides of a respective one SSDT device 1800A-1800D. The use of the integrity bands ensures that the TFL assemblies 1870A-1870D or the seal has not been compromised or opened since sterilization.

Once the one or more the SSDT devices 1800A-1800D are taped and banded for integrity, the set of SSDT devices 1800A-1800D are delivered to the autoclave 1920 or sterilization chamber (e.g., autoclave) using the autoclave carriage 1910, at Step 1732.

FIG. 19 illustrates the delivery and rolling of the transparent sterilization, storage, display and transportation (SSDT) system 1900 into the autoclave 1920 from the autoclave carriage 1910 in accordance with some exemplary embodiments of the present invention. The SSDT system 1900 includes four SSDT device 1900A, 1900B, 1900C and 1900D. The horizontally stacked SSDT devices 1900A and 1900B provide a first layer. The SSDT device 1900D is vertically stacked above SSDT device 1900B. The SSDT device 1900C is vertically stacked above SSDT device 1900A to provide four SSDT devices. Each SSDT device can sterilize, store, display and transport trays with instruments in a closed and secure container.

At Step 1734, the set of SSDT devices 1900A-1900D are rolled into the autoclave 1920 or sterilization chamber. The wheel assemblies of the SSDT devices 1900A-1900B horizontally stacked serve to assist in the rolling of the set of SSDT devices 1800A-1800D off of the autoclave carriage 1910 and into the autoclave. The wheel assemblies also allow the set of SSDT devices 1800A-1800D to be rolled back onto the autoclave carriage 1910.

At Step 1736, sterilization of the set of SSDT devices 1900A-1900D takes place in the autoclave 1920 or sterilization chamber wherein steam or sterilant agent is permitted to be saturate without air flow. Alternately, the steam or sterilant agent enters from the top and is pulled through to the bottom of the autoclave 1920.

FIGS. 20A and 20B illustrate a flowchart of the sterilization process of step 1736 in accordance with some exemplary embodiments of the present invention. The steps of the sterilization process may be performed in the depicted order, in a different order or one or more steps may be performed contemporaneously. At Step 2010, the sterilant agent or steam is introduced or imported into the autoclave 1920. The sterilant agent or steam will enter the interior chamber of the SSDT device through the vent openings or ports (e.g., vent openings or ports 185) at the top of each SSDT devices 1900C and 1900D, at Step 2012. The interior chamber, instruments and trays within the SSDT devices 1900C and 1900D of the second layer are sterilized, by the sterilant or steam, at Step 2014.

At Step 2016, the steam or sterilant agent is infused into the SSDT devices 1900A and 1900B on the first layer through the top vent opening or ports (e.g., vent openings or ports 185) of the SSDT devices 1900A and 1900B. Additionally, at Step 2018, the sterilant agent or steam existing or pulled out through the bottom vent opening or port (e.g. vent openings or ports 110) in the SSDT devices 1900C and 1900D is infused through the top vent opening or port (e.g., vent openings or ports 185) on the SSDT devices 1900A and 1900B, respectively, where the interior chambers of the SSDT devices 1900A and 1900B are filled.

At Step 2020, the medical instruments and trays within the interior chamber of SSDT devices 1900A and 1900B are sterilized by the steam or sterilant agent. At Step 2022, the steam or sterilant agent is channeled out through the bottom vent openings or ports (e.g., vent openings or ports 110) of the SSDT devices 1900A and 1900B.

The autoclave 1920 may have a drying cycle to dry the fluid of the steam or sterilant agent. Therefore, during the drying cycle, at Step 2030, the air or heated air will enter the sterilization chamber. At Step 2032, the air or heated air is infused through the vent openings or ports (e.g., vent openings or ports 185) at the top of each SSDT device 1900C and 1900D. The interior chamber, trays and instruments within the SSDT devices 1900C and 1900D of the second layer are dried with the air, at Step 2034.

At Step 2036, the air is infused into the SSDT devices 1900A and 1900B on the first layer through the top vent openings or ports (e.g., vent openings or ports 185) of the SSDT devices 1900A and 1900B. Additionally, at Step 2038, the air existing or pulled out through the bottom vent openings or ports (e.g., vent openings or ports 110) in the SSDT devices 1900C an 1900D is infused through the top vent openings or ports (e.g., vent openings or ports 185) on the SSDT devices 1900A and 1900B, respectively, where the chambers of the SSDT devices 1900A and 1900B are filled with the air for drying.

At Step 2040, the medical instruments and trays within the chamber of SSDT device 1900A and 1900B are dried by the air. At Step 2042, the air is channel out through the bottom vent openings or ports (e.g., vent openings or ports 110) of the SSDT devices 1900A and 1900B.

During the sterilizing step, the autoclave 1920, in general, infuses the autoclave chamber with a sterilant agent or steam for a steam or sterilizing cycle. The steam or sterilizing cycle is generally followed by a drying cycle. The SSDT devices 1900A-1900D are subjected to a steam cycle followed by a drying cycle. The steam cycle may be 4 minutes. The drying cycle may be 40 minutes. Nonetheless, other cycle times may be employed and may be a function of the autoclave operation.

The filter is made of a material that permits passage of sterilizing fluids while excluding passage of microorganisms. In an exemplary embodiment, the single-use disposable filter includes a temperature sensitive indicator embedded within the fibers of the filter. A portion of the single-use disposable filter may automatically provide an indicator such as a color or image responsive to the sterilization temperatures.

In an autoclave where the sterilant agent or steam is forced, the steam or sterilant agent is pulled through the top vent opening or port to the bottom vent opening or port through the shelf assembly and trays and into the lower SSDT device immediately below the top SSDT device. The bottom vent opening or port in the top SSDT device is aligned with the top vent opening or port in the lower SSDT device which provides a path for the sterilant agent or steam from the top SSDT device to the bottom SSDT device. The forced air pulls the sterilant agent from the interior chamber of the bottom SSDT device out through the bottom vent opening or port thereof and out of the autoclave.

In another exemplary embodiment, there is no forced air with the sterilant agent or steam, the steam or sterilant agent saturates the sterilization' chamber of the autoclave and falls under gravity toward the bottom the of sterilization chamber of the autoclave. Thus, the steam or sterilant agent enters the interior chamber of the SSDT device through the vent openings or ports. Specifically, the top vent opening or port on the top SSDT device receives the sterilant agent or steam which falls under gravity. Thus, the sterilant agent or steam under gravity would transfer to a bottom SSDT device through alignment of the top vent opening or port of the bottom SSDT device and the bottom vent opening or port in an upper SSDT device stacked above the bottom SSDT device. The sterilant agent or steam falls under gravity from the interior chamber of the bottom SSDT device out through the bottom vent opening or port thereof and out to the autoclave.

Referring again to FIG. 17B, the autoclave has a plurality of cycles of complete sterilization. After Step 1738, the set of SSDT devices 1900A-1900D are removed from the autoclave 1920 at Step 1740 using the autoclave carriage 1901. The handle bar in the SSDT device 1900A may be used as a handle for personnel to pull the set of SSDT devices simultaneously and in unison onto the autoclave carriage 1901.

At Step 1740, all CI and/or other indicators should be inspected as well as the sterilization tape and filters. In the exemplary embodiment, the filters may be configured to change colors based on the sterilization process.

At Step 1742, the instruments in the trays may be inspected before leaving the sterilization area to make sure the proper instruments for the scheduled surgery and surgeon are fully assembled and ready for use. The transparent properties of the TFL assemblies allow the trays and instrument to be inspected in approximately 360 degrees when on the shelf assembly. The trays under the shelf may also be inspected.

At Step 1744, the protection filter covers 139 (FIG. 2) may be placed over the top vent openings or ports in the TFL assemblies of the second layer SSDT devices 1900C and 1900D and over the bottom vent openings or ports of the CBFC assemblies of the first layer SSDT devices 1900A-1900B. The filter covers are optional. The filter covers simply provide an added layer of protection to the filter especially during storage and transport. The vent openings or ports covered by the stacked arrangement of the SSDT devices 1900A and 1900C or 1900B and 1900D protects the concealed vent openings or ports.

Thus, personnel may visually inspect the CIs, integrity bands and the instruments before transporting the SSDT system or separated SSDT devices to an operating room or storage area without opening the TFL assemblies or compromising the CIs, integrity bands or seals. The ability to visually inspect instruments without compromising the CIs, integrity bands or seals will serve to minimize delivering the wrong or insufficient set of surgical instruments to a particular operating room and surgeon. In the know systems the filter wrap or integrity bands are compromised in order to take a look at the instruments.

Before the SSDT device(s) are removed from the sterilization area, filter covers are installed over the vents or ports. Thus the set of SSDT devices may be stored.

Sterilization phase 1512 is followed by the post-sterilization phase 1514. During the post-sterilization phase 1514, personnel will transport the visually inspected unsealed set of SSDT devices to the operating room or storage area. The SSDT devices can be visually inspected when sealed and locked in the operating room or storage area to check for the correct set of instrument trays without compromising the CI, integrity band or seal. When opening the SSDT device, the integrity band is removed and latched unlatched. If sterilization tape is used, the tape is removed. Thereafter, instrument trays from the SSDT device are removed.

The SSDT device permits ingress and egress of a sterilizing agent but bars entry of microorganisms which could contaminate the materials and instruments stored within. The devices are configured to maintain the sterility of the trays, instruments and other items until the TFL assembly is removed.

While the present invention has been described with respect to various features, aspects, and embodiments, those skilled and unskilled in the art will recognize the invention is not so limited. Other variations, modifications, and alternative embodiments may be made without departing from the spirit and scope of the present invention.

What is claimed is:

1. A method for sterilizing a set of medical instrument trays with a set of instruments, the method comprising the steps of:
linking, selectively, transparent sterilization, storage, display and transport (SSDT) devices together wherein each SSDT device includes a transparent lid assembly having an interior chamber;
loading said each SSDT device with a portion of the set of medical instrument trays when said lid assembly is removed;
closing, sealing and latching each loaded SSDT device individually with the transparent lid assembly; and
sterilizing, simultaneously, the closed, sealed and latched SSDT devices linked together wherein the portion of the set of medical instrument trays in said each SSDT is visible in the interior chamber of the sterilized SSDT device through the transparent lid assembly.

2. The method in accordance with claim 1, wherein the sterilization step comprises:
filtering a sterilant agent or steam entering through a top vent opening in said lid assembly of said each SSDT device; and
filtering the sterilant agent or steam in through a bottom vent opening in a base carriage assembly of said each SSDT device.

3. The method in accordance with claim 1, wherein the linking step comprises:
hitching a first SSDT device horizontally to a second SSDT device before the loading step, wherein the first SSDT device and the second SSDT device include wheels.

4. The method in accordance with claim 3, wherein the linking step further comprises:
stacking a third SSDT device vertically on top of the first SSDT device, after the lid assembly of the first SSDT device is closed, sealed and latched; and
stacking a fourth SSDT device vertically on top of the second SSDT device, after the lid assembly of the second SSDT device is closed, sealed and latched.

5. The method in accordance with claim 4, wherein the sterilization step comprises:
filtering a sterilant agent or steam entering through the top vent opening in said lid assembly of the first, second, third and fourth SSDT devices;
filtering the sterilant agent or the steam through the bottom vent opening in a base carriage assembly of said first second, third and fourth SSDT devices; and
filtering the sterilant agent or the steam filtered through the bottom vent opening in the base carriage assembly of said third and fourth SSDT devices through the top vent opening in said lid assembly of the first and second SSDT devices.

6. The method in accordance with claim 5, further comprising:
covering said top vent opening of said third and fourth SSDT devices; and
covering said bottom vent opening of said first and second SSDT devices.

7. The method according to claim 3, further comprising:
tailorizing said first, second, third and fourth SSDT devices before said linking step or said loading step.

8. The method according to claim 3, further comprising:
banding a latch bar of said each SSDT device with an integrity band, after closing, sealing and latching step.

9. The method according to claim 2, wherein the linking step comprises:
stacking a second SSDT device vertically on top of the lid assembly of a first SSDT device, after the lid assembly of the first SSDT device is closed, sealed and latched; and wherein the loading step comprises:
placing a first number of trays on a shelf of the first SSDT;
placing a second number of trays below said shelf in said first SSDT;
placing a third number of trays of a second shelf of second SSDT device; and
placing a fourth number of trays of below said second shelf of the second SSDT device.

10. The method according to claim 1, further comprising:
installing a first disposable filter in the lid assembly of said each SSDT device; and
installing a second disposable filter in a base carriage assembly of said each SSDT device.

11. A method for sterilizing a set of medical instrument trays with a set of instruments, the method comprising the steps of:
selectively linking together two or more selected transparent sterilization, storage, display and transport (SSDT) devices of a set of SSDT devices based on a number of trays in the set of medical instrument trays wherein each SSDT device includes a transparent lid assembly having an interior chamber;
loading said each SSDT device with two or more medical instrument trays when said lid assembly is removed;
closing, sealing and latching each loaded SSDT device individually with the transparent lid assembly; and
sterilizing, simultaneously, the closed, sealed and latched SSDT devices linked together wherein at least two medical instrument trays in said each SSDT are visible 360° in the interior chamber of the sterilized SSDT device through the transparent lid assembly.

12. The method in accordance with claim 11, wherein the sterilization step comprises:
filtering a sterilant agent or steam entering through a top vent opening in said lid assembly of said each SSDT device; and
filtering the sterilant agent or steam in through a bottom vent opening in a base carriage assembly of said each SSDT device.

13. The method in accordance with claim 11, wherein the linking step comprises:
hitching a first SSDT. device horizontally to a second SSDT device before the loading step, wherein the First SSDT device and the second SSDT device include wheels.

14. The method in accordance with claim 13, wherein the linking step further comprises:
stacking a third SSDT device vertically on top of the first SSDT device, after the lid assembly of the First SSDT device is closed, sealed and latched; and
stacking a fourth SSDT device vertically on top of the second SSDT device, after the lid assembly of the second SSDT device is closed, sealed and latched.

15. The method in accordance with claim 14, wherein the sterilization step comprises:
filtering a sterilant agent or steam entering through the top vent opening in said lid assembly of the first, second, third and fourth SSDT devices;
filtering the sterilant agent or the steam through the bottom vent opening in a base carriage assembly of said first second, third and fourth SSDT devices; and
filtering the sterilant agent or the steam filtered through the bottom vent opening in the base carriage assembly of said third and fourth SSDT devices through the top vent opening in said lid assembly of the first and second SSDT devices.

16. The method in accordance with claim 15, further comprising:
covering said top vent opening of said third and fourth SSDT devices; and
covering said bottom vent opening of said first and second SSDT devices.

17. The method according to claim 13, further comprising:
tailorizing said first, second, third and fourth SSDT devices before said linking step or said loading step.

18. The method according to claim 13, further comprising:
banding a latch bar of said each SSDT device with an integrity band, after closing, sealing and latching step.

19. The method according to claim 12, wherein the linking step comprises:
stacking a second SSDT device vertically on top of the lid assembly of a first SSDT device, after the lid assembly of the first SSDT device is closed, sealed and latched.

20. The method according to claim 11, further comprising:
installing a first disposable filter in the lid assembly of said each SSDT device; and
installing a second disposable filter in a base carriage assembly of said each SSDT device.
